SEQUENCE LISTING (24 sequences)

SEQ ID NO: 1 - DNA, 44712 bp, homo sapiens
agtaaagttgaactcacatagaatgagtaatttcttataattttcacatacatcaagaagtttagttaacgtaatcatttttgtttgttcttcctttccattggaagcatccttggagatgaaaatgcaaacaaagaagtcaacttttcctgctatcacttacaatggcagaagtgttggttagagtggtgcagagggcagtgttcagagtacgctgcctcatcacacattatttgtgcttttaaaattgactatgtaccatggtgcctggtgcagaatgagtactccaaacttttctttttaatgtattacatcatcttcctgagaagtagctctattttcgccatgctcatctatcttccttccccttactaagatattaatatactgagattaaaactacaaccttctaactagtttccttgactctagtttcaccactctacatgtgcttgctagatttgattttctaaaataataattggatcatgttattacctcattcaattacctttgataacatctagattcaagcataataattggcacatattcatcaacaaattaatatttgatgaacaaatgaatgaataaatgtataagtgctccatgggagaatttcaagtccttccacagttagaccttaccctcatttatttcattatctccttttagttatttccataaacatgggacattaatttattgtgtgactttgacatgtcagataccatgagagttactgaatgttcaaagaaataaaggaactcaaggcttctttgaaagaaagtatctgcttaagccacattgatttatttattgtcttctgctttcccattgccaaggtttgttagtgtcattttgccaacataaaaagccatttctttcttttctctccatgcatattcttttcatccctcaggatttagctcaaattgtaccttgttcattataaacacttacctaaccactcaaatgtaaagtgatccatccctcaatcccttctctgactttctaagacaattattttaattacatcatactttactttggccgggaattatacattataagttttctggtatgctaaagctaaaatattattatacaaatctatagtttatgccatatctccttatgcaacatatttcccttgaggctatgcccttcacatcgtactttgtatttcttggtatcttccatggggcctggtataacacttcctcccacagtggttcctcagtctatattttttgattaattgattgatgagttattgtaaatttccttgtatttaacagagaggtatgtaccttgtgcagactaatggatattcaataagtgttgatttactgtccaagtacaaactaatcaatattatggaaataataattaatcttttaattaattacaactgacaaaagagatgccccagagcattcaagaaaagtcaggaaattaaggaactgaatatctccttgatactttttaatctattttaattcattacttatcctttccctgtctctattcccatttctactttactgctcagagtagaaattcttaacctggagtctctgtttccttataaagtccatgaatggatttcagctggacttggagcccaataaaatgttatgcataaagaaagccaggctgttatgtaacaacacaaagtcaaggcttaaacgaaagcttatctagctccaaactatatcatagcttagctgatttcttctgttgttaggcagaaaatccactggactataaaagatgtgcagtctggttattgtactggagtgtccaaggtaaagtttgtaataacaaaaaacacaagagaagcagcagcggaagcaacaagtgttgcttgagatgtgtcaggcacagttctaaatgttttatgtattaaacttctttgaacctcactataaactcatgaaggggataccactgtggctactcacattgtataggttaaaaacctggagaacaaagaggttaagttacttgtacaaggtaccaggacaccaaacacaatggtaagggatggaggaatagaggcatgggaagatagccaaggtaaaagttgtctacaccaaattttctttcaagctattctgtattcaaagtgccctcagttccacgctagtcatctttcttggctagggaagaactctgtctacagtggcaactacagagccagacaattcacaccagacccagatctgactttgcagagtcagtctacagacaaaccagtcacatcaggattgtgaagataatcacttgcctctagacctcttagaaagtaaaggggtgtcagaaggagacctttctaagctctggcattcttcatatccttaatgggcaggtacaattaaatactttgtatgtgatgtcaatgagagagaatggccctgaaatgaacttcgacaatgtgcagaatacagttaaccctgttagctagtgggttgtcagtagaaatcaaagtttaagtagtttaacagtagatgtgggatgtgctctgaattggaatatttcacatatctatatgtcagccagggattcagggaatcctaccccaaacattccaagccagaatgactttaagcaatctgtgcacagtgctgtttctagtcttaaaatggttccatttcatgttggattgcaagagacctggtttctggtcatagtgtcatgacctcttggtgacttaactgcgtatgactttgttgggacatagtttcttctactgaaaaatgagtatgacgatagatgtaacttgtgattaaagatgcagcttttaaatatgatgatgtttggtaaaatgcttgagattttggaggtgagtttctttccaaacaacaacaacaaaaagaaaatttttatttgttcctgaacaaattaaataaagttttaaaagcattgcaacttggtcatggtttgtaaaatagtctgaaaagatgttatctcaaaactttgataattacttttctatatgtaagaatttgcttcctctttcttaagatacattatagtttagtatagtttagtaatgaaaagtggattaggcatgagaagacctggatattagtttctgctttgtttcttgccagctctgtgaacttgggcaagtcactgggcttcaattttctcatttgtaaaacagaaatagtaaatatgacttatcttgtctaactcaaaggattgttttgaaatatgtcaagacataataaaagcttgcatttacagagattttcacttataaggattaagttctgagttaagtgttttatatatattgccttatttaatctccactccaacactatgagttaggtaagaatttgtatccatttttcagatatggaaactgaaattcagagaggttaagcaagttgcctgagttcatatagcttactatcggggggaaggaacaagatctaaatcttaaccataatggttctgcttttataaaagtaaaattatctacataaaaaggcattatgtctttttttctcctcctcctcatttttcttcttctactttttctttaatacttccttttctctctctctgtcttgtaatgatggccataatttgtcatatacactttagtagggttaagttttgataaacagtaagccaggattggggcaaaacaccagtccgggtacggtccatagccaatcatttgattgtctttactaatttagcaaatgtatcttggtaacttagtatataccatggctattctgggtgcagggattctacaaacatgaacacaatgtcattcctcccttggagtaagacacgtacataaataattataatatgaggcaggagtgattatatgtcatcaaaaagagataaatgccatataaggagagttcagaaaggaaattaataaattccaagtaaaggggatcatggacttttgcaccaatgtccagggacaatagtgcttgcctttgtctaaactatcagaataactagatatgtggttattaaatgttaaggagatgcagagtagggtaaattgtttatctgagaggctgggcttaggaaataaggcaaactataacgtggtaacacgcctccccctccttagataccacttgagaatttaatttctctaagtttttttgaggaaaaagaatatttactgtggaagaatatactttgatggctggaggagggaaggaaggaggctggaggatgggagggggatcatctcatgtttcaaactgttgatttccagtttccacctggtaacagcctaggggccagagcataatgatacaagtcaaaacacagagacctacacagagacaactaacagcatccagcaagggaattataaaagctcaaaaaagatttccagtggctaaataaatacctttccttgtagttgcacttgtttttcttttcatttactgaaatttggaaagacacatagactctattgccttttcaaagttctagtttacataaaagtataatctatgaaaatcaaatttttcatatatagaggatgaaactatgttgtgatcaactacaggttatctatgtattttatttaaaaactggtggacagggagaaagattttaaactatgattaatgaaagtgatagatgacatacttttcatataaaaccatgactttctttcaggacactttagaaatatgtccaaaatatacagaaaaactccacagcatagtgcttgaatttacacaaacattccaaggcaagtaggaatccgaatgagtagtttcacataaggctactcatttatttttgcagtccacatttaaaacaactacaagattaatttcattccatttttctggacttacacatcccaaaagcaagaaaaaaatgtcattttggaagtataataaatgctacttgtaaacaaggctagaaagggtggagaatgaaatgagatggggggtggggaaacaaaccctttagttacaatatcttagggaaatgaatctacaataagacaaatttcaaatcaagttgctccactatactgcataagcagtttagaatcttaagcagatgcaaaaagaataaagcaaatgggaggaaaaaaaaggccgataaagtttctggctacaatacaagagacatatcattaccatatgatctaatgtgggtgtcagccggattgtgttcattgagggaaaccttattttttaactgtgctatggagtagaagcaggaggttttcaacctagtcacagagcagcacctaccccctcctcctttccacacctgcaaactcttttacttgggctgaatatttagtgtaattacatctcagctttgagggctcctgtggcaaattcccggattaaaaggtatggttttcaataattgtcctgaactctgctacagactaataaaacttcggtcaagttgcgagcaaaagggattatttggtgagaaaaagaaaaggaaaaaaaaaactgttaaacttttatagtttttatgcaacattttaaacaagtattatttgaaaacagaagttaaatatttgataaaatgattgaatgcattgttttatttctttctcttctgtttttgcaaagatgcttcttaaaaaactgtaacctaacaccatgactctctatattaatgtttttgtgtttgtattaatgaatgtttagctcttcaagagatggtaaccgaaaagtattctgaagtagcatatctgtaattgacctatatatttaaaaaatgtatttttctcctgaatttttgattgctttaaggacttttaaaagcccagaagaactgaactctggtgattagctgaaaatacctaatgtatatttcttcaaccccagaaatgaacagaaattatcaccagagaagtgaaaataaacattgaaaatatttatgttattaaatgcctaagatactaaaaacgctgtataatttcttaaatgtaaagtgtcccagtcagtatcttcactgcaagtgaaattttaagatacattcactataaaatgaggatgtgggttaattttctaattttaaaacagggtaacacagtgaccactgattaagtgggctcccagtgatgtaaatacctgtggcttttttttttcctgttgctgtttctaaccagaaaagagccaaatgcctcctatagtaacccttgtttgtggataaatgccgcctttattctttattttaaaattacatatattataattgaagtttttaatcatctagtgctgtaggattttaattttgttttgcatttatagcttcacttatctcttgagggttaaattttgactttgaaagtgacaccagaggcatagataacataatgcaaatgttgatctggatgacatacttcatttaatgggcttaactagttttaggaaccagaaattattatttttggtaaaacatttgtaaactatggcaactagcttcagtggagtagtgatattacattagtaattagcatcttggacattgtcatgcatttttagactacttaaaacagtttcagtataatttaaacagcatatcatctcaataacaatctctttggttactacttggacagctgctctttgctctttatatttctgattaattaaggaaagaaggaaatgacgaaaggaattatcagtattgacagcccacattaaatgttttaagatgctgggtcttgaagtaccatgacactaatttaaaacagatgattttatggaagattttgcacaaagtttctggacatgcaattattttttcacagtacagtcagggttaaatgcacacacacacatacactctctctctgtcacacacacatgaacacacacacatacattctctctctctcacacacacacacacacgagcaaatgaaaatgaaataaaggtcggtgattgaagttataagctgcctgtctatattcagcttcaatcaaaaagaatattgtttggagaatttgtaccattttaacaggagtaaacaaacataaattataatactatttctctcagctaatactcaacattttctttttgtgggttgttagtgaaacaaccaggcatgttctgctctggagaaaagatttcaaagtgacatctgcattaactgtcgttgctatttattaaatctgcaatgactttgcttcatttttcttttaaacttgtaagacattaaatatgcaaggctagcaagcttgtggtaaaccataaacattgcatttttaatgggaaacagatacaaaaccactccctcagatacaatccaaaggaacaggctgccaaaacaaaagtcttctcttaaagggatactcagtaactgaatgagagtgggctgctagaagaagcttaatgttgaacttaaagtggcagtgtgaataataatctttatgatattttgttaaatgaaaagccacaagcctgtattcaacatgtgctgccttagacacatatcagtagtaaatataaactaagaacaaacatgtaagaaactgaaaaacatttgtttcttctctagagaatctaggcaaaaattcccagaagataccttaaggttagatcatacaaagatctttcttttctcttctcttctttctttctctctctctctctctttctttcactttcttcctttctttctttttctctttctttctttccttttctttcttccttctttctttctttcttccttccttctttctttcttcttttttttaatatgctacaattggataatgactcaaaacagctgtaagattctacttacgaacgtacagctttaatatggattcgtctaagtgtacaattcaaggtagagatgatgggactaaatatctgctatgatgggactaaatatctactatgttttggagatatcagaatggtttcaatgcaatttaaaaaatatttcatctcccaaatgactttgctgctatataatcaagtacttttgttattcagatttattacgattttgtcactcagatttatttagtgcacttcagttgatttaacaacatagattcgtgcttgtggaatgaatggataaatatggaatacatttatcaaaacaatacttgtttttttacaaaatgccctgttagcaaaatgagatcatatatacatcacataaaaatcagaattttccatatgtgtttattagcttattaaacataatgtttgagaaactaggaaattgatttattttttaaagccgatcttgcttataccaagttgaggttttcaatctgctttttttatatcagaaattcgatactgaaaaaaagctaattttgtgttttaaaaaatcagactgattgcttggaacaaatggaataaatcgtcttgtcaataaagtgatggtcaattgagtcatttgtgtgcaaaatattgtgcaaggcccgggtacagaacaaggaataataagacacgccctgaaggagtacatcgtctagtgagggacagaccaagcacgcaaaacaaattgcaatataatgtgataagttctttaaaagaggtaagagcaacgtgctttgggagcagagaagagggagaaagcagcatcttgcctggatgagccaggggacacagaagagaagcccactatctcatttaatctttacaactctcttgcaaggtcggtattctacatggcaagtagctttgattgtcagggttcaggtgaggactgggaatggagtgacaatgaaaaggaatgtggctagagctattaattcataccaatttatgagtggtctgacatacagcacagggactgtggacagtattgataagccataggaagttaataaatggtttatagtagaaattaataagacaaaattgaatttttagacgatgactcaggcagcaaaatggaagatggactgaaatagaggaaatatgcaaggcagagatgaattagaagctcttgtagtagtttagggagagatgagaagggcctggatcaaaataattgtggtaggatggagagatgtgactggcttccagagatatttctgaagcagaaattggagatctcctatgtggatagagataattttatctgaccatgtgttaagtgagcattgaaaagcccaacatgatttattaccatgacaattttgcaaatgatttggatatatagtatatcatcttagtttagacccaaaattcaaacaagcagatttaaataatactcagatttctcatcctaagtaaatagctgtgcaaatgatactataatgtacctgattgtctttcaatacatttttaatgctctgcttcctgataacaaatgaaatattattttgcatcttttctctgtgctcactgtattgtggcatattatataagaaacgacacacagattaggcagtgctactcttcttaaatctctcatttgtttcctaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtgagtgggatgcagtttctctttacctaccttacttgttttattcttgggaatggcaactctgagagtttagcattttcagttttaatctctctaaatttgagagacagtgccacctaatgaggagtaaaggaagcagaacaggatagaaagaaaagtagcgttgttgttctctctttctctctctctctccctctctgtccctcttccttttttcctccattactttgcaatcttggacaaatcatttttaaaaatatttctgagctttatatcccttcttctacaaaataaaattttgaattaaaaaagtcttatgttctaaaatttcagagtaatggtttaatcttaattctataatatgcttttataaaatattagatattaagaaaatcaggtcagtctctctactattcacttagcccagacaacagagtttcaacattactaaattgtaaaataatcagttatcaaaagcaaaacacatcaaattttatgacaaaatcttatctttcaagtatcaaaatactcccttaatctctgagtcttagttttgtctttacttttgacattttgtaatccagctgcttatattctttagaaagtttcaaaggaaatatttatcactaaggtatggtgtaaatattcaatataattgtgttacatatatctttagcctaacaccctaaagacagtttgatttatcatcttttcagaaaaatttggtctggaaattccaagagcattacacttagaagataaatctccagaaagattgaagctcagtggatgggccaaaggggaatgaactcagtttacatggcagctcattcacattcctccttttgaaggggtgtattttcttactggagatgaaaagaaaaaaaaatctttatatcatgattccgccatgtgtacttccttctggaagaaattccatactatgatcaggcacttctttcagaagtcctatatttactcagaacatgctttctagcagtagtttgcagaagaaaaatgcaacttctacaattggaataaactgcctataggaacccagaaaaaatataaagtatattcagtagtaacgactgaaaaacacttgaaatagcttcttcaacacatatattgtatagctatttaaaacccaaatacaaatgttatatgaagactttcaaaaggccgcaggataattccaatggatctatttgttcagtcattcagaagctatatcgggaacccatcatggatgagctctgctctaggcatggggagaaaggatgaggaagacaaatatggccctagttcttgtggatcttatgttctagtggtggtgactattactttttgaattgtgtatttgccacaggggagattggattactcttcagaaattttataagcatctagagatgacctgcattattgccttgggcaggaaaataacagggttgaaaaaaatctgaacaaagaaatcaccatttgatggatatacacaggataatcttttctgaagttactaaaatggcgaagttcccaaaaaatgtaatgcgaattaaatttcactttgcctctgagaaaaagttaaaagaatttattattactctcgctacatgtattatcaaaaatgaagggttgatatttttaaggaaagaagagttggccagtcactttagtgaagttttattttttcgtaaatatagtttgcctttgcttccatgaaaaaatagctcataaaccttgaccctgatgaaagcactgggccagttttccaattctctgttatgtaaaaaggcataagtcaattgttcaaagtgaaacaacaacaacaacaacaaaatgtagtcttaggaagcagcaagttcactgacttgggatctttatgacagttttgttgttgccattgatattgttttgtttattttttgttttcagatgagaaagttttctacatgttatcttttttctaggagctcaaagtgtacatcattcctttattatagctaggtttactgactcatatactaaggaagtagctaaaattataaaaataatttgtttttaaaaccatatttaactaagggaactaagtaagttccaatgagcagtggtctcatgcaaggtattttcaatattttaaaatttacagatgaatatttaaatatattataaaagttttaatcagctatctctaagaaaatacatttcttaaagggaaatgaaattcacttgactttaaataaaacaaatgaactcatttcatgtttttaactattatctaactcttccttactttatgatgctggcaagctgttgagagccttgacatctccatctgcagaaaaatcacagtcttagaaatcctattaatcgtgtgaggtacctgggtcatagtagcagcttcatgcagtgttaaaattatatgatgattatatgcagtaacagatgaagaaaaaaagaaagaaagcaggagaaatgcaccacctcattcattgtaaatgcagtatagttgattttttaatttgttttatgtcctctagtgatctaagcatgaagcttgaattattataataaagaaaataaatgcaatgcagttggggatggcaaatgttaatgcttatctgtatcaaagactaacactgtcttcaggattatccttggtggattatccttggcagacacttaatgagcagagagaagctacaatgttgaaggacaaaagtcctttgtcatcttattatcgaaataatgtttaatacaaataaactttttaaattaaaatgtttgactactttttaccactaagaaatattcttggcaaagttaaacaacatcaaaattttaaacaattctttcaaagctaagaagtaaaaacctatgaaattaaaattatgtaattactctgcaggataatttgatgcctctggaaaaatgcatggactgttttagtccctaattctaatttatcacagtagtattaacacgtgcttttttttttttttaagaggatgagggattgagacagtaaacatgaattcctcatagatattggccaagttgtttcattgaacttgttgcaacttaccttgtaattggaaccacaattttatgagacccatatcttgtcttcttttatctatcatgttaagaaataaaccagttccctcttaattatagcatttatttcaaaaagcaatgttaaaaataaatgtagagttagcaaattcagaagtctttcagttactttataataataaatccacaatgtaactcattcttctaaaaatcaaaaaaatttaaaacgttctgattttgatgggattttctattttatatgcttggcatcctgaagaagacagcttcccttacttaggtactctttggcttagccattcatctttggatcaagtgtcaccaggtagtataatttggtatggacttaacacctcattactgaaatgcaaatcagtcctgacaagtcaaataagaacaagtattgtcttttgactagtctccaaaattaaaataataattcttgatgtatttcttataaacaaatttcagaagagttaaaatgcctcttaaaactatttaatagtggaaagaacttgggctgtgcagttaaacacctctgatctcaaattctgctttgctgtttactacctgtaaccttgaaactttcaaaactttgtttcctcatttgataaatggaaatatatcttatttcactgggtaagaatagaaaaatatatgcacaaatgtttattatagtaacttacatttcacttaccatttcaggcaaagttggctaagaacatgaggctgtttctgatatttccttttatttttatgtattaatgagaataataggtataggccatatacttattagagactctcaatgacttatacttattattctcaattcatattatatcatattatactcattgaaaataagccatatacttattagagacttatttagagtcttattagagacttattaagagtctctaataagtatatgggttgtcaagctagacacaaaatagtaccatctctctccacctctctaaagaggaccacatcatctccaaatgtaaatagacaagaacaaaaaaaattgtgcatttactaggcaattgaatgttctccaaaccaatattcctttgaacaaaatgagtttgtttgatttgagaacaatgaagtatttggctattttataatcataagtgattcatacccccacacacttagccctctagaaatggtgccagaagatgaaacacatttaattttgccatatagaagcattgcaaataactactggtttctaataatgtaccttataatgcaaataatgttttaatttttaaaaatatcagaaaaagtacaaccttttaaattatgtagttgcttcttcttcataatcatttttcttgaactggtgaaaaattttgtcagaagttaccacttgggacatattgtactacagcttctcgcttggctggagaaactggttttccagaagcagaccttatacaagaggtaaatgaaccattagacttctcccatgaaatcaacccagaaaaataaattaggactattttgtagaggaacagacagaatgctagaggctcagttgcctttatcatttatgtctggctctgcagcagcaggggtgtgaggtgctcctcattcagagtttcgaatgcaccaaaagggtgtttccaaaacatgatatgggaaggaggctgtgcacagagtatgaccactggattgcctaaattgacattgggtaggagtggagtatgggtgatcttcagatgaagcaagagtaatgctcaatgtgacctggttaaacaagtctcctgaatccaggggttaaagatggagaggaggaagagagggagtcaagaaggtcaaagggagcgtctgtctccctgacctgaatgacaactaacatagcccttgtttccttcttggtccccaggagggagagagaggcagctgtgtgggatcaaggcattttgtgagattgcagctgtgattcattttcctttggaagaacatccctagctccaggggatatggtgggaattggttagctaaaatatcaattccctgggtaaaagaggaaaaccatgagaccctctaatcttaacttttctccttcaaaagagtggaaaaagattacatagttcctcaaaaattaaaaaaaaaatgagtctgcaattcagtcttaataaaaagagaaagcccttagcatatatactgtaacagtaaagactcagttactcttgacttagtctgtggactgctttaacttctgtagcttgcctacttctgccttttttactgtaatctacatttgctttgattattctataataatcaattatatatacatatctgtgattatacaatatcttttatctttttatatatctattttattttttttcactagaaaagaagtattgcaataatttatcttcgtacaacctctacacaacaaactgccttatactttgtagactctttacacatcactgaacgattgatgaatgaatacacgaacatatgcatggcttggttcttgaattaatgcttggatgaatattctacaatattcttgactctagcaagctgcaaagagaagggaacatgcctgattgagaaacaaaagtgatttctcagtgcatccttgctccttccctttaaggacaactgtaaaaccaggaagcttagaaattccttgagtttgactggaaagagaatagaattaaagagaagaatcatctatgtatgaagcctggtctaactgataacgcagtcagattgccatttttaagatggaaaaaggggaacatgcttgccttgagcagaaaagtttttggtggggctggagctcctaaatattattttggtcatttgcccaaaaactgaagtacaccaccaatgtcccaattatgaagttgaatacagaattataaaaaaaaattgttgaaagtcaataagaataaatcttagaatttggactcctgccacatggcagaaaaaaagcatgatgttttaagacaacctggcctttgtccaacatgatgtttaccagattgctgacattttctagcataacaatactattcatcattgggataaccaaaagaagggaaataaaaaaaaaaagaagtctatttcttactagtctttattcagaaatgatttttttggtaatatcttaagtagtatcttatttgctttaataaataacttgattttaaagaagataaacctccttacaaaaactgggagagcaaatgtctaatccaggctaccttgaaaatagtgtgttttagtggaaaatttagaattatgttaaataaggctgctcaatgaaaaataaatgtaggaaaccactatgctcctgttactacagctgagtgaattttaccccaaaacatggtggcttaaaaaaaaatttaatttgctcacaacttgaggtatggaaaatttgggaagggctctgaagggctgttctcacttggggtcactcttgcatttgtagtcaaatgttacccgggatttataatcttaagaaggctagcctgggctggacacctgttcactctcataactgacagttgctgctgctgtcgcccacaagcttggctgttgtatcctctggattgcctcagacagcctctcttttttcctatggcccctggctgtcccagagcaagcatcccagaattgggtagcagctgtacaaccttttctgacctagtctcaaatgttatggtgtttcaggctattgattcctagaggcatcacaaggccatcctgaattcaaagggaagagagaggaattagatgctacctcctaatggtattgggataaatgcatattgtagaagagcatgcaggatagaaaatttaatgtatgccatctttgaaaaatataatcatctacatatttacttacatttatatacatttctaatgagaagtttgagcataattatgtattatgtagagaaagcccacagatattacatgtcccccatgaaggtcattatttaatgattacaaactacattaagatgttaaataattataattaaattaatttacaaattaacttcatagttacttaattcttctacttaagattgtgagaaaagtattaagtcgtaccttaatgactttgttttcattatggaacattttttggacataaattgaacctggctaattgatacatataatttaaaatgctaacattttactttttatatcattttatgaataaccataaaattcatcagtatcaataggaattatttagttaaaaatcacatacaattaatacaatcataggcttaaaggcagaatttaaatttgtttatttaaaagttaaaaaaattaaggtaggataactgtttcatagaatactatctgtttttatattttttgaagattaggtttagttcaaataagctttttgcaattatcaaaaataatctctggtcctcgtgtatttctaccacaatttacttccggcttggtgttttcagatgaggaaatggagcctgaggaatgctaacttacagattccggtcatctctccttaacagccaagttgattttaacccgagccattctgactctaatgatgtacaaacaaagagtaatattttatagatttgcaaaagagtgtagcatttgctaccattaaaatagggttaaattgaccaggagcagtttttgaggagtctaattactcattggggatcatgtgcttcctaaactgaaggcttatccaaaagccctgaacttggtagacaagcaccccaaaggccctgaacttcatgggaacaatgatgaatgcagaaagaccaggaaataactgtcaagacaaaaacatttcaggaccaagatctttagatgaacaccttgtgcttgacatacctgaaaaacagatttcagcctctaggataatatgcatagtaattgtacagaaaagaaagagttacagatgaataatcagaaagattattcagaaggataatgagagcaacaccctctattttgcttattaataataatcataggtagcaatacttgagacgctgctatgcactatggtaggagattattttgtagcatagcttattgaattctcacaaaaattctgtgaaagacttaagaaaactgaggacagagggtttaaacaatatgtcttctaattttctttttcttttttttttttttttagacagagtcttgctctgtcacccaggctggagtgcagtggcgcgatctcggctcactgcaagctccgcctcccgggttcacgccattctcctgcctcagcctcctgagtagctgggactacaggcacccaccaccacgcccggctaattttttgtatttttcagtagagacggggtttcaccgtgttagccaggatggtctcgatctcctgatctcgtgatccacccgcctcggcttcccaaagtgctgggattacaggcgtgagtcaccgcgcccggcctcaatatgtcttctaatcttaaagataaagaagaggaaccagaattgaaattcacataacagtcttataaaaaaatgttattataggagcatgtttaaatatgaaaattctgcagaataaaatgaccccattgggtacccaccacagagcttcaaccagtgtcaactaatgccgttatttttctatctccacccacttccatatcattttgaagcaaattccagatatatgattttatctaacaatacacttaccagtatgtttctctgaaaaataaagctctcaaataaaatagccatagtttatagcttatttatatgagtcaggatccagatgaagtacatacattgctttttttaaaaatagcttctgttttttggtttttttttttttttttttttttgacaggatctccccctgttgcctaggctggagtgcagtggcatgatcacagctcactgtagcctcaacctccccaggctcaggggatcctccttcccgagtagatgggactacaggcttgtgccaccatgctcagctaattttttgtaaagatggggtcttgctatgttgcccaggctggtcttgaactcagaggctcaagcaatccacctgtctgagattccaaaagttctggaattagaggcaagagccacagcacccagctaaaacatctcttaactctcctttacactacagattcacaatttcttcattttcttttggaatttagaggattaatcagattcaggattaattgtttgtttttgaaaatacaaatctatgaatggggatttgtttcttcatttagaagcttctaactgatgcttttccaggagaaatatatgccatgtagagaaactcttactgctggagtagtcactctattagacagaaaaatcagtcttcttgagatatgtatgtgacaaaaggtgcttcacaaatatggcaaagacactgaagaaagtaagagcaattttcaggtgcagataatgggaatgacatatttgatagtaagtagcattcatttataaaatattcctggaaacaggaaaaaatataattattataaccagttgtatcctaatacttctgaggaaaataaaaactcctatgatagcaggttatagggagatggttataacagaacatggcagaaatgatctcaaagccatactcataaacttttcatcatgggacagcaaggcagatagactttaaaattaattaatcagaaaggcacttcttgttttcaaagcagccagagcaaaatgctgatgagaaacagtaattagtcctccttactcaggaaaaaaataaataaactaacaaaacaaacaaagaccctgaaatgcaggacagtatagtttaaacttatacatctatggcctgtaagaagtttagaggtgcaagattactgttatacaggggtggtcagcatgaatattacagaggagagggataacggatatgcttcaagggattgttagaatggaaatagaaataaatgaataagtggagttttacataaaatatgcattgcatgaacaaaaattgagtagctaggaatgagtgtaatatgctcacggaatagctgtagatcagcattagaaaatatgcacaatgatcacatgcataaatggcacaccacggctaggcacggtggctcacacctgtaatcccagcactttgggaggccgaggctggtggatcatgaggtcaggagatcgagaccatcctggctaacacggtgaaactccatctttactaaaaatacaaaaaattagccaggcactggtggcaaacgcctgtagtcccagctactgaggcaggagaatcgcttgaatcgggaggcggaggttaaagtgagctgagatgatgccattgcattccaggctgagcgacagagcaagactcttgtctcaaacaaaaaaacaaaaacaaaaacaaaaacaaaaaacatagttctggagtgaggactcaaactatcctggttcttgatctactttagccatgaggcaccttagataagctatttaacttctttgggtttctgtttccttacctatgaaatgagaagattagactgccttaaaattctctgatttctgggataagtaaaattttgaatgttgaacgagttaatttctcattaaagtcaataccaaacctaacaaaagaagagcctgccatctgctatcataatcattttttaaagaaggtgtatttaaaagtagaaatggaagtgaaggcaggaaatggtctcagatggtctcagaaattgtgtgtgtgtgtgtgtgtgtgtgtgtctgtctgtctgtgtgtgtgtgtctgagataatttacattaaaactaaccacactgcttttattgttttttttttattttgatatggattgctaaaaacctcatcatagagtctacctaatcagcatttgggaaccatagattagctgatatttaaggacccttccctttgtgacattcagagaactcataataactagagtaaattcttcattatactctgtctcttgtctttgagcaagctagttaacactccaatgccttatctaccccttacaggtacagcatattactgggtctgatgtcagaacaattatttcaggggatacagataggtatgccagaaaaaaaagtcatgctggaagccaggagagttgtcatctaatccacattttgtcattcctaactagataagcttgttcatgttatgaaactttttaaaataattgaaagtatgagattaccatactgccttacctatctcattgggttattgtaaggtaaataataacatattttcatttttaaaaataaactctaaaactaaattattaataaaaaatatgtgaaatccccccaataaacataaatttactaattcagagcaggttgtagaactataattttaatcattgaaatgactctccaagaactaaactacaagaattagaatcctgaggatggtattccttttttctgaatctctagaaataagatgtatataaattaccttaagtagttcctagtgttataaaattttaggatgacaacataagtgaaagtgtcagccatccagacacatcattctttccctacacatttgaaattttatctgcagtttcgcattgacaaaatgttgagagtcactagaatttaagttcgccaatgaataaaagcaagcgtaagtggaaattaggactgtgatgaaggtgcccaagaaggaatgataaaatgtagtaactacgttccaaatgaaaagaagtttgtcttttctgggttggagaacaccaattaacaaagatttggcagaaatctaccattcctaattgcatgaaattagcaaattgacatttccgaaaatggcagtgattacaactgtacagtgtcattaagcaacctcctacaaggtagctgttctggtgttttaccgttcatcactgaaaacacttaactcttgttatttctatgggaactgtgtctataagagttacacatttatgtaacatatttatgtaagttcagaattacttgtattttgatacttaaacttataaagataggtggtcactgaattaagaacaagattccacctgcaaccccaactatcaatggagcagtgatattataaataaaaaaatatttggaaaagactcagaaataggtagcttcaggtacgttttctgtaactttcatggaagaaatgaagacagtcaaagtatggctaccatggtggtttacaagtatgtttacataaaactcaaaattgaaaagtgattttctcgacatagcggttattataccctgttgtaattttattttaattgaaaacagtaaactgaattatatttttatgaagtcacgggcaatatctgccttggtcctgctatattcccaggatctaataaattcatggtgcccaagaggtattcaataaaggcttgttgaatttatgaaaaaataatcttcagtacttcaagtttcagatatgatggagagattgcttctaccaaaggctaaaaaatatttctgatttgtgttatgcagtcttaaaatacagaaatgatcacattaatttgagttaatagaatatcaaatttttacttgatgtcctacttcaatatgaaacaatgaaaaaaagaaaggcaaaaggccttatttaataattttctctattatgattcatgatgatttattcaaaaataactgccttttgtataattctggttcatccttatatgactagctgaatgactactaaaatattttttaataccaggaccatttggtaattcaatgaatttacatttacaaactaaaaacaataatacactcagctagaccaaaatctaaagtccaaaagtaatgcttcataaaattaatttcagatatgtcatactacccaatttatttcattctaactagccatatatctattggtcataaaatatcaactgaaatggatattgttttggcattccaatgaaaactaaaagaaccgttatcccaaaatgttggataaattttcaagatcctgccacaagttgattatatgtttatatgattttaactgtagatctgtgacagggttaggtgtatcaaaaagatctttattactgatgatttggctagagtaattattgacacggtatttatgtaaaaatgtataaatttgccaatcactcgtgctgactacttatcaaaataactacttttcaatacaacaacaaagaggtaaaacatatagatttgagccacaccctaatgcaacacaaatgttcccctaatgcagaggaataattccatgaggaaaatatcatgttgagtttattattaaatacaaaaagtcatttgacttggttgcaaaagataaagcaaaaattttaccctacaatgaatgtaattcttagaagaagaaaaatatgttgttactgttaagatgatcatcataacattaatttcttggtaaatatatgctgtgtgattctgtagccacatgtcctttctactcacagcacactcctattactcttatgctaggtctggacaaagtgccaaaggatcttccccctgacacaactctgctagacctgcaaaacaacaaaataaccgaaatcaaagatggagactttaagaacctgaagaaccttcacgtaagaactatttttttttaactcacaataagataagactcaaggtatttttcaaaacactacctagataaggccaagagtactatgtaccaacaaagaagcacattagtgtttattaacttcaccttttttttttttttttactttttttttattaattctgccccaaacactgtttttctttttctttttttttttttttttgaattatgcttcttttttttttaattttttttttattatactctaagttttagggtacatgtgcacattgtgcaggttagttacatatgtatacatgtgccatgctggtgcgctgcacccactaacgtgtcatctagcattaggtatatctcccaatgctatcccttccccttcccccgaccccaccacagtatgagtgagaatatgcggtgtttggttttttgttcttgcgatagtttacctttttagcagtggtagggaagcaaagcatcatatttattaaccactaacaatttacctgacttttttaatacatataccccatatatcttaggtattttcttcataaaaatactttagatcttaattctctctcacgttttcaggaataaaaattgtctttcaaattaaattgtattttaatagtggaacaaaacacaaaatggattgatcctagtattattttatttctttgtttcttttgtttgttttcatgcattttctaagagaggtgtgaatgatgttctaaagtctcaaagattggaatcaagtgaaaaaaagataattattcatatttcatttagcgaaatataattctatcttcatatatatgtttcttgctttatctttaggattatgcatgctgtgtccttatctgtatgtttattttgattttctcttatctctctactctatcataatgttcagtctatcagtcaggatttcatcagaaaagagatgccactcacaaaggggtttcatggaggagactttattgaaacctctatttgcaaaggggcatgggaacccaacaaggcactgtgaagcacccagtcccacaaaggtctagaagaatttgagggtctcagagccatgggagagctgggtagttccacctcttaatttcctgcctgtgtcttgtgtggttgcatctacttcagaccaaaagacaagggcatttgattaatgcattccccagaggtcagcttcccaggacttgaagaagggccaaaaaagggcaaagaatgaatattgtggtagttgggagagaatatgctatacaagggaaatgaccaacccaataaaatagaggtaagccttgctaagagaaccttctgtaacagtaaatgcattgtttgttcacacagaacaccagaatcactataaaatgcattttctcacccagttctctatctgtagaatgagtgtgatgtggtcttccatcaaaatatgagtgcacattaaatataaataaggcagacttcaatgtccaaaatatatactctggaaatctcactgtgtctacttccattctggtagaccactctgaagcattgagtctggttcttggcattttaagaaaggcattcaagaacatctagagaaggatgaccatggagataaagaatctggaaattaggtctcataggcaccgtggatggagaggtggatgtctaaactcagtgttgagggtcactagtggcacatttgaagggccatgttgaagaaaaagaatggtatttacttgtttcaactacaagatgacagaatcctcctagtgagtagaagttttacagaagcagatttttattcaatattaaaaataaaaattaaagaatcagaattttcagacaatacagagacttacctagtgatttgttacctgcctgtggagattagagttcacagcttcgtataatgaatgtcataggaatacattcttaatgttgggagcataaacataccaaactctaatttcatatctacttatttattctagtcattatagaaaataattgaggaagatatgtcatgagaataggcactacttactaaagtataaatataaatcatctgatatacattagaataaaaatatagatacaaaggaaataatataaaatagaataaactggtcataattattacagagttactataatgaagataccatttagttataaactttacaatagccaagatgggaaaagcagtaaacatttagccattagaagaaaatataccagatttttttaaaggaagaaaaatttttttctaaaattttaagactctacaaggaaccattttaaaaaatgcagaaaggccgggtgcagtggctcatgcctgtaatcccaggactttgggaggtcaaggcgggtggatcacaaggttaagagatcaagaccatcctggccaacatggtgaaaccctgtctctactgaaaataaaaaaattagctgggcatggtggcgcgctcctgtagtcccagctactcgggagtctgaggcaggagaatcgcttgaacccaggatggggaggttgcagtgagtcgagatcacgccactgcactgcagccctggtgacagagtgagactccatctcaaaaaaaaaaaaaaattggtagataaaacagtgccatgatcaatttattgacgtgtccaacaatataagcacaatatgatatgagtagtgagtgttatggacttaaagtaaaagaaaaaattaggcatcattatttatccaattaccagacacagttttgagagaatatgcgaggaaggagttttagacgatcagcagaagtttgaaggactttctgtacaagaagaaagcaaaggcagaagaaaataagaaacagacaaataaaaatacgagttggctagagtgaaagaaacagctgaagcaatggaggataaggacaagctggaggttccgacctttgggtctctttccgctgatgagaaccttattcaccaactccccctcacttcaccttaaggtttcaccgggaatgagctgatggggaaaccagcaaggttttcagtgactatttaagctactgttggcataagtttaaaaaggagaccataaaaaatgaatattattaaatttagattactaaaatatttttaaaacttttatttggataagtttgccttatagctactgttttaaaggatgaaacattcttactgtcatctcaacttcctgtacatttaagtatacttggaatattttttaaaaaaaccctttatatccttaagtaagtgacatctagtgtccatgacattctcattaaatctggcatttgtaataatgtgttgtttcttttcctaagactccttctgtttttaggcagccatcatcttgacaatctctttctttctacttcaaagtatacatcagatgaataaagagagttcattagagccttcaaccattcagaggatgttaagcagctatttttctcttactgtttcattcatgatcctttctatccttgttattttgcaaataaacatcataatttcacctagcaaagccacacttaaggaaggagagaaatgaaaatttagcaaggattaaaatagaaagaatttaacagaatcatgttccctgtatgtgcttcattatgagtggtgtgtgttacataagagtaattttatacctatatatagtgggaaaaactatataattgttctataaaataatggaaaattaagtgtggtaatgacaacatttaatatattttctttgaattgaagcttaataagttgagtaaggaaaatttttttaattaaaaatcagcaaaaattgtcaaagactgaacaaaagcaaacacttgtttagcaattactatatatactaggcactattcttagtaccttatatacattaacttatttaattcttataactattcttatgaggtagtcacacttgatatctcctttaaatgggcacagaaaggttaagttacatgctcatagtcacaggccaaataagacccagagcttgagattctaaactcagaattctggcttgagtatgtgcactaataattgcctataaatgtaacagtcaccaaaaaagcctttagaacaatatttggtacagtgtaagacctacataaatggctttggagaaaaaaatcatattaaaagtcaggttttaaaaacatttaacataatacagatttttcagatcatgtttaatacatctaattatatcacactttcaaaatggtcttctatggataggaattgaatatctcctttgcgattttcattaggcatccacaattttggatgtcttgtaatgatatatgtcacctcactccactcagctggctctggcttttccctgattttttgtttgttaatgtcaatttttttatgaccttaactcccacaagtttttgaacttttgattattaatcgcagtgtgttagaattgtgtatcaatcaaccaatgcatctttcaggattagttaaatgattatgttacttaataagaagttttaattcatcgagtaatgtgaactatatgaggatatacatatattaattaatcagctattacctttagaaaacattcttgcatttctgagactaaaaatactcgctttcctattataggtccccttttgcccctccacattttcatatatgcgaaacatatactataattgaaaatgcaaaattagaccttaataagttattttaagttccttataattagttaataaagtggcttgaaataataaaaccatgtggttttgttagacatacagataattgaaaaagaataaaatagtccaaaaataggcctagcaattgttataagcataataataatgaattagaggtaatttcttatataaggagaactcagaaataaaaatcaaataattacccaaaatattaacgtatttgtcatataaaacctttacacataaaaaaaggcaaaaaaaaatcaggagaaaaatatgttaaggctctctgtgtcatattgaccatatctttattttctaaatcaattattaacctatattaaaatttaataaacatgagaagtattggtgatcaagcaatctcctctctccaaaaaatacaataaatggtaggaaaataaaaaaaaaatagtttaacctttttaaatgacaaggaaatacaaagaaaagcaatgagctacaacatgatattattttggccatactcttaagaaacataatatcaaatattgcaaggctacgggaaaattttagttccttgcattgctggtgatagcataaattggtatcatccttttgtagcggaatccatgttcatgatcaatgaagatggacttttaagttaaaaagctgactgcgggatggatgtctgtggcattgattattttcaggtatctagggtcatttcaattgttagcagtatttattatttaaaaattacatagatatatacacgtacatatacatacatacatgtctgtgtgtgtagagtgcatgtatgtttatgtatatattatatatcttattacatttcccttctcttcattcctgattttcaggtcctcatattataactttttttttttttgagacaatctttcccagtcaccaaggctggagtgcagtcgtgtgatctcagctcactgcaacctctgcctcccaggttcaagtgatcctcctgtctcagcctcccaagtagccgggactaccggcacccaccaccacgcccagctaatttttgtatttttgatacagacgggatttcgccatgctggccaggcttgtctcgaactcctggcctcaagtgatcctcccacctcagcctcccaaagtgctgggattacaggcatgagccaccgcacctggcctatacacttcttaagtaaagaaataatacttacttaatttttttctgatttcttgcaattttttagatttctctgctatccctatgaatggaatgaacaaacatgcccttgttttacatgtatgtggaaggttttggatttaaagagatgtttttcttcactatctacaggcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcataggtacagacatttttataactttaagaccaaaactcaagtttttatcttaagagatttcaactgggttttaagtgcaggaaggtaaattagctacaagctacatgcctggagcctatgtgatttataagaacagggccaagagaaattgggattttgaatctggaccttacagtgatgtttttatgtatgtccttgagtatctgagaatatggagaaaaccctagggctgaagttaccctaccacacacacaatatgcaaagatgagtttaaatagtgcttaactacagatattttgagatacctttccgttttggggacctgttgtattctaaacactcataaatcatttgctttaaaaatatgttatggctgggcgcggtggctcatgcctgtaatcccagcactttgggaggccgaagcaggcggatcacgaggtcaggagatcgagaccatcctggctaacatggtgaaaccccatctctactaaaaatacaaaaaattagccgggcgtggtggtgggggcctgtagtaccagctactcgggaggctgaggcaggagaatggcgtgaacccgggaggcggagcttgcagtgagccgagatggccccactgcactccagcctgggtgacagagtgagactacgtctcaaaaaaaaaaaaatttctctaaacatatagtaaaaattgtaattttattattggcttataaaatgctagattgaaattctttaacaaaattttaaaataccttaggaaaataataaataacgtaagatgggaattgtaaacttgctttagttgatttctgggcaaacctgtgtatgcatcttaaataattttattggtattgctttcactaatagtctttttcataagtcattattattcataagtcattgtctgtttggcttttgttttaataagggaaatttttatcctattttgctattctcctgaggagcatgctctttaacttcacttttgttttttagtcaagttgatattaatccctatttctttataggttgcttgctgaaacagatactacagtgactagtaatatcctaaaatttcctctaaaagccttgcacttattacatttgtagaactgggcaccaatccgctgaagagctcaggaattgaaaatggggctttccagggaatgaagaagctctcctacatccgcattgctgataccaatatcaccagcattcctcaaggtgatagaagattctccaaaacattttgaaaaatttaaattcaaaggctttaattaaaaaaaaaaaatcttgtggaaactggaattgtcacctaagaggccaaccttatttccagcaaattctctattactgctagtgtttcatccagtgtttatatttccagcaaattctctattactgctagtgttatatccagtgattcagtctggatgaccacggggccatccaggctcactgtagagatgggtagactcctgttaatgtctagtttaagacacaacttaatgcacagtgattttataatattgtaaaatcacaaagaaaaagtacatttatttaaaaaatagcatgccttggtgtttgtaaaaaaaaaaaaaaagatatatgggagtacctctgaaattgatccaatttttatcttgagtctgcaatagcttagggaatttttttttcctttttaagacactccagttattaatatctgaatgatagtggtagttgtgtgttgtgaagagtgtagattttggagtcagtttgcttgagcccaaattaccataaagtggttcaaagactttgaaaaagttattcatttgtcctctatctctttatctgtcaagtggaaataatactgctatccacttcattgagttgttttaaagttaaataaataattagagaaatatttagcatatagataagtagggaattaatataactctctattatgcattaaaaatgttctgaacaatcagtatgtgtaggcgatgtaccaaacacttaagacagggcagggaacaggatatgctacatgcctttaggagcttactctctaaaaccaagccagacaaatacacagcaatgaccacatagccacaagggtaagttcattgttccaagggattcccaggagaggcacaaaagccagtctttgtgaattacaaaaggcatcaaaccaccaataataaattcctctaacttgcacatcgtccctactctgggtttttcagtctcacagaagccctctatattatctagttttcccctcagtctgtccctttctcaacttagatgccttgttcttgagtgcgtacaaaatgtagacatttggccacagaaaacaccagttgtcctttattgttcttttacagaaatttcaactcctgagagtacatattccataggatgctttcttaccaccccttgccctcctcctagtcttttgaatacagagcatgcttttgacttccttagtagcaactactggcatagatagatacatagatgataaagactgatttataattcataaagcactttcatgttgagtctccaattttttgcatgtaacaagtaatcttttgctatccccagtgcaggaatgaaatatttatatctcagatagatataaattggggattgatatagatagatagatagatagatagatagatagatagatcttattaaaattcttgctgaaatttatttcatgcagccagatgacttacatttagcacaatctcagactttgctgtccctcttaatgcatgctccaaaatatccttcactgataccttttatcatttcatgatctcttttcctttactttgctatgtcttctatctatcccaggtattctttcctcatcatttatagcttgcttttatttactaccgcttaatagaaaagttaagtaactacaatttattaagcatgtatttagttccgggtatcatgccaaatgcttgtcatggagtcttctatatgattcttacaacgtccctatatagtagggttataattcctgtttcataaaagtggaaaatgaggcttaaacaggtaaagaaaattgtaattaagttcttagagctaggaagcggcagagatggaattgtgtggtgctgatgtcagaactcgtgtgtttaactatttcactctgttggcatattaagtcctcctttctattcatcgttatgcttatctaatacatgattccaccttgcttaggctgggatacctatgcctgaatatcagcaacctcctggtgatattttctcccatcactcatggaaatggaaaggcagtagaaagtagtgtagagactataaaaatcatacacttagatcaacctgggtgagagacctaactcagtcatttagttgtactgtgactttaggataattattctgcatttctataacctcaggtttgttagatgttacatggagctattaataaccctgtctactaggattattgtgaggattactctatagaagtaaggcttgtattgtacacgcaagtacttggcccaatgtaaacctcaatatattttatcttaaaaaaggaaaagaaaaaagaaaatttaaaataaatgaacaaacaaataaataaaataactggagtatgggttctttaactcaatgccacagttgcagaagctaatgatgcactttacaaatttgtctatctcaaacctattgcaaggttttctcccatagtcaaacttcttcttagctataactttacatttgggaaactgacatcccaaaaagggagacactttgaaactaagtaaggccgatggttgtgatagcaattggtgtactggaggcactgcattaaaaaatatacattttgcattactttgtaattactttgtaactttgagtcttatttatttaactgtgaaaagggattagtgaccaataatttatgttttgagaggcttaaaggatttgaagtaggaaaagtgccaaggtcaatgttcggtgcgtaacagacatacaataaatagatgttccttttcctagcagctctaacacaaaatatgctaccataggaaactaagaatcattttcaagtcaaatatggactaaactattttattaaatggacaatattgacagaattcagtgtgatgtgataaaaattttagacaaaatgaaacctgtggtttttcaattctgtgtctttatagacaaatagccaagactttctgccaatcagatgttctttctacacttaaccaatttgctattttctctgtatctattgctattaagtggaacaatatgttattttaaaagctgtataaaatataatacagcctcagatgctaggaggaaatgaaaataaaatatttaaacttttatatttataatactcgatctttggaaatcacagccatgcaagtactatctagaggtttagtgggtactttggatgtaaaattattaccccaggtgctccacatatggcagatcacttgattatagacctctccactccttctgcataaacattctggatttaataggcataagtaaatgttagctgtttctacacagacattttgcagaggtatttgatgcttttatggaaaagtcaattaaggctaagaccagcacagtatatcagtgttaatttttaatgatgctcattaatgaaatagacaattaagtttgtacatgggtttaatttatacgtaatctcttcattatggttcgaatagtgactttcccactttataattactatcttttatcaacgtcagttttagcccattaacaaaggtggaagagaataatgagactcagcgttgacttcagttcttctattccaagaacaaaaattgactacatgatagaaatcatccaggtttttgttaaagtcaacctaagagaaaaatagcaagggcttccactacttccaaaagcacacattattataaaagggatgaaaaaaaaagataaaagtaaagctgattctttgaaactgtgtgtcttttagaaaattgatggcatatgtcttctttaagtttgtccacattctgtacttataatgtttatctgctaatttaacatctttaattccacattccaggtcttcctccttcccttacggaattacatcttgatggcaacaaaatcagcagagttgatgcagctagcctgaaaggactgaataatttggctaagtaataatattctttcatgacattctatcagaacaatggctaggtatattagtgcttatatgataagaattataaacatgatgattgatttttgagtgtactacatttcactgctgttgttgtttctataatctcaaacttcagcatatccaattatttcttgagaagaagccaatgaaatagtgcccttactaaaaccaccaagatcagcttcatattttattaatactatactgtttatagattagttatattttatacacaaagtgtgtgattaaatcaatatgtgcatttaatatgttctaacttatttgcatcaaacagaaagttaagtaaattataacacagttgctagaatttgaaaatccattaaaatgaaatctaccaagacagagaaatattgagcatcacacaaattactattttaataattatgagattctctaaccccctcttctctgaaaagtttaaaataaaatatctctgggcatttaatgtagatcaacaaaagaaaagcttttgttgttgttgtttttgctggcccttcttaatgattttttttaaaataagtgatactagtacttgtcctggtttcaaacataacttcaatctggcaaacgttttcacttgtcttcaaggaacactatgttctttttgatctgtacatattgctgactgtccctacagagaagcacactatcatgccagatttactcattcctgttgttatatataaatttcatggtggataaataaatataaaatatatctttgaatgtgtatatatatatatatgcatgtgtgtgtgtataatcaactcctttactaggtaaaatattctttgatgggaaaagtggtttttgatccatgagacctctgtccctcacaatgcctagcataatatcatgcacataagttttcaagaatatttgtttattaaatatacaaataaatgtttgaataagtgaacagtttttaaaaataaactattaggaatattatgttccaatattatggttgaccataatttgatgttggaacttaggatattataaggaataggagactatgtccaatcacgttctgtcagatgaatctcaaagacagtaactttgagccgagaccttaacaataaatggactttgtccaggctcagagaggttggagaaagcactccaagaactgggaagagtaagtgcaaaaacaaaaaatgtcctttcctatagacttgattaactacacaatgcttgtaagaaaggcatccatgtgtggtgtttgctttcatttctgctttctgttgcaggttgggattgagtttcaacagcatctctgctgttgacaatggctctctggccaacacgcctcatctgagggagcttcacttggacaacaacaagcttaccagagtacctggtgggctggcagagcataagtacatccaggtaatgcaaagccactgcttatgtgaggaatatccaaaaagcaagacccccaccctgcttatgggatgctgggaagcaggaagctcttcttagaagttttttttccagagtgtcttagttagaatttctatgacaaaataccataaaaaaaattaaatcatgaagcacaatttacattagctagatttcaataaagtttttgatttagaagtatggagatgaaacttaaagtcttctgtaatgctgatatttttttcagtaggggtggtgatgttggtgccatagtgagtagggggagtttgaagccacaagtaaaataaaaattctttttttttactgtcactaattttaattttcaaattattcaagaattgactggaaagagcttctttggtctagctcctgattctttttgacatggcctcatcatttttctttatttcctctaaaaaaaaaatgagatacgtttgcagaatgtgcaggtttgttatataggtatacgtgtgccatggtggtttgctggccctactgacccatcctctaagttcgcccgctcaccccctaccccctgttgtgtgttttgttaccctctctgtgtccgtgtgttctcagcattcaacttccatttatgcgtgagaacatgcagtatttggttttctgttcctgtgttagtttgctgaggatgatggcttctagtttcatccatgtccttgtaaaggacatgatcccattcctttttatggctgcatagtattccacggtgtatatgtaccacatttcctttatccagtctatcattgatgtgcatttggattggttccatgtttttgctattgtaaaatagtgctgcaataaacattatgtgtgcatatgtctttatagtagaatgatttatatttctttgggtatatacccagtaatgggattgctgggtcaaatggtatttctggttctacatccttgaggaattgctacactgtctttcacaatggttgaactaatttatgctctcaccaacaatgtaaaagcactcctatttctccacagcctcatcagcatctactgtttcctgactttttaataatcaccatactgactggcatgagataatatctcatctccagccacagggaatcattaatctattttatcatttttgtttgtttcatagaccatagttttgtgtagattccaggaatactttaacatctagaaattttataattttaactcctgtatttggttttataatctaattttgagataatcctagtatatgatatgagtctaattcattttctgtatgtgatagcaattgtcccaatgtcatttgttggaaataatattctttctccaatgaattgtcttgtcactgttgaaaatcactcgatcataaatgtaagggtttatttctggactttcaattctattacatttatctatatgtctagtcatatgacagtattatacccttcaattactgtagcttttgattacgttttgaaattagaaaatgtaggttctccaactttgttcttttttttcaaagttattttggctattctggttcctttgcattccatatcaattttaggatctactttttcatttctggaaaaatattcttgtaaggctttgaaaggtattgttttgaatctacagatcattgggaagaattactatcttaataatattgagccttccaagtcacaaacaagggttatttatatcttcttttctttttctcaacagtttttgtcatttttagcatacatgccttgcaattcttttactacattcattgtttttcattctttttgatgctgttttgaattgttaatttttttatttgtttgctgctaatgtataaacatacaattgatgtttatatattgagctatcctggtactatgccaaatttgtttttagttgcagtaatttttgtgagttcctttggatattctatattcaggatatattatttaaaaatagacagctttagttattttatttggattcttttaggtctccccctcccccacaacccaccctttgcattggctggaatcgccagtgcaatgttgaacaaaggtgatgaaattagacatctttgccttatttgctagtttaggaggagggagcattcattttaccattagttacgatgttagccacaggtcttctatagatgctctttatcaagttgaggaagttcccttctatttctgatttactgaaaacatttattatgaatttttttcttacatgtttttcctgcttctattgagataatcatgcgagttttggcctttatatttttgatatagtgtattacattgatattactttgattgattttcataagttaaaccaattttacaaccctgagataaatcatgtttggtcctatagcatgaaatcattttatatgttgtgaaatgtagcttcctaacattttgtgaaagattttttgtccatattcataagaggtatttgattacatttttttttcttatgatgaattttgtctggctttggtcttagaaaatattgaccccatagaatgagtttggaatctacttttattttgttctacacagactaatacttacgtcttgatattttgttctctcactttcaatattaaccataaattgctcaccaaacaccccttcctttacttgctaatcactactggaatatagaggatggtaaatgacaacaagaaacttccaaaatatgagaaccataaaccatacatataagtgttgctttcactttaatattccttgtttctgtttttatatgacatctactaattcgcttttcagcccttacaaaaacaaagccgttttcaccagcacatgaatgtgaatgtcctagtgggtagaacaatatttcaaattttttttttttttttttttttttttttgagtcggagcgtctcgctctgtcacctaggctggactgcatacaatggcacaatctcggctcactgcaacctccgcctctcgggttcaagaaattctcctgcctcagcctcccgagtagctgggattacaggcacgtgccgccatgccatgctaatttttgtatttttagtagaaacggggtttcatcatgttggtgaggctggtcttgaacacctgacctcatgatccatccgcctcggcctcccaaagtgctgagattacagacataagccaccacgcccaggctttcaagttatgttttaagggagtacttcataggttttatagctcacttaagatcttgttggtggcaggacgcagtggctcatgccagtaatcccagcactttgggaggctgaggtgggcggatcacgatgtcaggagatcgagaccatcctggctaacatggtgaaaccccctctctactaaaaatacaaaaaattagccgggcgtggtggtgggcgcctgtagtcccagctactcgggaggctgaggcaggagaatggcgtgaacccgggaggcggagcttgcagtgagccgagattgcgacaccgcacttcagcctggataacagcgcgagactccgtctcagaaaaaaaaaaaaaaaagaccttgttgtcttagtgttatattctaaccttgcttgtatgatttcagagaagttgcttcttgacctacagaactcatatcctcaaagtgaactgtaaactcatttctaacattctttgtataattccggtttttagggaccctaagaaaatctatggccaaattaccattatcttaaacatgattgtaaaacataattcagaaaaaaaactctgattaattatattgcactgtattcaagtaaaacgtattacgtaaaatatgtatttgttaaaacttcctaggtctattctaaatgctgtgatgtttaaatatttaagaatttcttggagtttgtgattctttatttcaattccagtactttatgctctctttttctttttcactacacagcactttctcattctatatcattttgttaaaaggtgtatatattttttgtcctgaaaacataatatttcatgtagacccaatatagtcatgtagacctgatatatgaaataacatttattcattcaacacatatttattgaacacctggtctgttctagtactggagatacagccataaacaagcagaaccattcttgtctcatggagcttttactctagaggagagaaacaaacaatatcaaataagcataatatgctacatattgataatgctatgaagaaaaacatgtaacgtaattctgccccagagaatggcagaatgggatggtcagggaaggtctctctggtaagaaaaaacgacttacatgaggcaacaagccttgaagacaccttagagattattccaggttgagtaaacggtgttgataacttaagaagagatcatgctttgtgcattagaagagcagcaagtagaccagatgactggcttttacagtaagagaaaaggaggtcagagtagaaggatgtcagatcatgtaggatttgtaggcccctttgagaactttagattttattattctgcatgagattggaaaccactggagttctgaggcaaagaacatataatttggtttactttctaaagcaattgctctgagagcttaaaaaaagaataaaatttaggaaggcaaaaggggaagcaggaataataatcagggcaacttcaatcatttggaaggaaacaatggtgacttggacaaggttgtcggagtagaggtggagagaactagattaagtgtacattttgaaggctgagctagcaagatttgttgttgattatgtagagagcataagagaattggagctcaaggataatgacaatgtatatagcctgagcatctgaaatgattggcatgcaatttactgagatggggaagacaagactaaagaagggaaaagcaaattttcaaaaggaagggagaaatcaaaagtttaatctgagaagtagtcagttattcaaagtggggcctgtcgtggtggcttacgcctgtaatccaagcacttcaggaggctgaggtggccggatcacttgaggtcagaagttctagaccagcctggccaacatggtgaaaacccaactctactaaaaacaaaaattagccgggcatggtgtgtgtgcctgtagtcccggctactcagtaggctgaggcaggagaatagcttcaacctgtgaggcagaggctgcaatgcactgaggtcatgccactgcactccagcctgggtgacaaagcaagattccatctcaaaaaaaaaaaaaaaaaaaaaaagtgaaggattaaaaaataataaaaaaaataaaatgtttacctgaagggcctcaacatatttagagaataataattattttctatcgtttcatgttgtaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattactattgtatacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaatattaaaactatcatgtgacttcatgtaatcaggctgaacatttctacaattactagatgtattagacgtaagtattttctttagttaaaccacccatgttagaaatgttttctgtagaatttataaacaactatcaatgcagacaatttaataagcctggggatgatttacttacagtaaacatttatcaaattgtacatttgtgctatcaacaattaataagcaaatatgtgaaaatagtttctgtcttctatgaagttagatatttgatggttaaaacccctataaatcatagtttcatatgggaaaaaataattgaaatacagtgtaaatttaaataatttattaagtatagcaaataattgaaatatggtggactaaattttgtcatagaaatatgtgcaagttatagtagtggctcacatgagaggtaatcaattctgctaatagtagcagaatgagtgcagtggaacatgaaaaacttgaggagataacagttgaggtgggtttccatagatgcataatagttcaagagcaagatttggtggggaggcactattcaagacagggactaagttcaaaatccaagacgtatgctgggacacacctctgacaggttggcataaaggaggcttaatcaaactatttttcttcttctgaaacagaagcaataattttcatttacatttgacatatcccgaggtaatattaacattagggaaagttactcttttccatctttccacattcttgcaggaccataaaatctgaattttccagtatttttaataagagggaagaaacctctctttttcttctctttttcatctcccaagagatcctcctctcatgactacagttgaataggtggtttctattggaagacattcaggaattcaaggtgcatgtccataaatggactttttttgttgttgttcagagctggaccttgaatgatgcatccttctctctgttgtaaccatgaataatgcacccttcatgctatagcctttaacgattcacccttcttattgtaaccttgaatgattcaccctttatggtgtagccttgagtgacgcacccttcatgttgtagccttcaatgatgcacactccatgttatagccttgaatgatataccctttatgctgcagcctttctcttatggggaaaagcctgcagatatcctgctgcttaactgacaagtgtggtgagaaataagtagaaatctaaagaggggaagaccattttggacacttatctgcaaggcagatccaacacacttttccagtagtcaagctacttctaattttgttcagtatcaaaatgagaaacaggcctgattctccagcactcttgtcaacacaacttccccccatatttatatatatacacacacatatatatctttatatatatacacacatatatatctttatatatatatatttatatatatatctttttgcatatatacatatatatgtatctttatttcctttgaaataaagataaatatagctgatttctttggcttcgacacttactatttgcatgactaagggaagctagttaacctttctgtgactcatttccttgtccataaaatgggaatattaattgtacatgtcttatggattggtgtgtgaattcagttagcgagtgtagaatataacttatagatcaaagtagagtaaatggaaagggctcaactatggtgttgctactgccattgttattacaggcacacagttcgagctataatcatttcaagggaaattcttatgtgtcagttctggatcgaggtctgagattctgcatttcaaacaaacttccaggaatgctgctgcttcttggtccacacttggagaaataagtcagcagagagtcctctcgtttcctattgtaccatgtctgtcttttgtctcctgcttattggcctctgtaaggaactcacagctgctataataaagtaccaaaaactgggtggcttaaaacaacagaaacttactttctcac

SEQ ID NO: 2 - DNA, 2305 bp, homo sapiens
gaatctacaataagacaaatttcaaatcaagttgctccactatactgcataagcagtttagaatcttaagcagatgcaaaaagaataaagcaaatgggaggaaaaaaaaggccgataaagtttctggctacaatacaagagacatatcattaccatatgatctaatgtgggtgtcagccggattgtgttcattgagggaaaccttattttttaactgtgctatggagtagaagcaggaggttttcaacctagtcacagagcagcacctaccccctcctcctttccacacctgcaaactcttttacttgggctgaatatttagtgtaattacatctcagctttgagggctcctgtggcaaattcccggattaaaaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattactattgtatacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaatattaaaactatcatgtgaaaaaaaaaaaaaaaaaa

SEQ ID NO: 3 - protein, 359 aa, homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 4 - DNA, 2151 bp, homo sapiens
ggaataataagacacgccctgaaggagtacatcgtctagtgagggacagaccaagcacgcaaaacaaattgcaatataatgtgataagttctttaaaagaggtaagagcaacgtgctttgggagcagagaagagggagaaagcagcatcttgcctggatgagccaggggacacagaagagaagcccactatctcatttaatctttacaactctcttgcaaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattactattgtatacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaatattaaaactatcatgtgaaaaaaaaaaaaaaaaaa

SEQ ID NO: 5 - protein, 359 aa, homo sapiens; identical to SEQ ID NO: 3

SEQ ID NO: 6 - DNA, 21 bp, artificial
  oligonucleotide primer
gaagaaccttcacgcattgat

SEQ ID NO: 7 - DNA, 20 bp, artificial
  oligonucleotide primer
ggaccgtttcaacagagagg

SEQ ID NO: 8 - DNA, 20 bp, artificial
  oligonucleotide primer
gaccactcgaagatggcatt

SEQ ID NO: 9-16 - skipped (8 entries)

SEQ ID NO: 17 - DNA, 20 bp, artificial
  oligonucleotide primer
ggaccgtttcaacagagagg

SEQ ID NO: 18 - DNA, 20 bp, artificial
  oligonucleotide primer
gaccactcgaagatggcatt

SEQ ID NO: 19 - DNA, 21 bp, artificial
  oligonucleotide primer
tgcattaaggggactaagcct

SEQ ID NO: 20 - DNA, 22 bp, artificial
  oligonucleotide primer
aaaagggagaactttagggact

SEQ ID NO: 21 - DNA, 20 bp, artificial
  oligonucleotide primer
tctgcagctctgtgtgaagg

SEQ ID NO: 22 - DNA, 20 bp, artificial
  oligonucleotide primer
taatttctgtgttggcgcag

SEQ ID NO: 23 - DNA, 20 bp, artificial
  oligonucleotide primer
acagtcagccgcatcttctt

SEQ ID NO: 24 - DNA, 20 bp, artificial
  oligonucleotide primer
gttaaaagcagccctggtga

(19) United States
(12) Patent Application Publication
(10) Pub. No.: US 2010/0273180 A1
(43) Pub. Date: Oct. 28, 2010

(54) DECORIN POLYPEPTIDE AND METHODS AND COMPOSITIONS OF USE THEREOF

(76) Inventors: Abhijit G. Banerjee, Durg (IN); Nyla Dil, Winnipeg (CA)

Correspondence Address: MUETING, RAASCH & GEBHARDT, P.A., P.O. BOX 581336, MINNEAPOLIS, MN, 55458-1336, US

(21) Appl. No.: 12/728,996

(22) Filed: Mar. 22, 2010

Related U.S. Application Data
(60) Provisional application No. 61/161,868, filed on Mar. 20, 2009.

(51) Int. Cl. G01N 33/53; C12N 5/071; C07H 21/04

(52) U.S. Cl. 435/7.1; 435/371; 536/24.5

(57) ABSTRACT

The present invention provides methods for decreasing expression of a decorin polypeptide in a cell, methods for identifying an agent that alters, preferably decreases, the distribution of decorin polypeptide in a cell, and methods for determining a prognosis for oral cancer in a subject through the use of a compound that binds decorin polypeptide. Also provided are antibodies that specifically bind decorin polypeptides and double stranded polynucleotides, for instance, dsRNAs, that inhibit expression of a polynucleotide encoding a decorin polypeptide.

This application claims the benefit of U.S. Provisional Application Ser. No. 61/161,868 filed March. 20, 2009, which is incorporated by reference herein.

### BACKGROUND

Oral squamous cell carcinoma (SCC) is the sixth most common cancer in the world (Jemal et al., 2008, CA: A cancer journal for clinicians, 2008 March-April; 58(2):71-96, Jemal A et al., Methods in molecular biology, 2009, 471:3-29). Oral SCC accounts for more than 274,000 newly diagnosed cancers worldwide, and are the most frequently diagnosed cancer in developing countries of the world (Parkin et al., 2002. CA: A cancer journal for clinicians. 2005 March-April; 55(2):74-108, Dobrossy et al., Cancer metastasis reviews. 2005 January; 24(1):9-17). Despite improvements in surgical techniques, radiation therapy protocols, and chemotherapeutic regimes (Cooper et al., The New England Journal of Medicine. 2004 May 6; 350(19):1937-44), the overall 5 year survival rate for oral SCC remains at 50% and has not significantly improved in the past 30 years. The vast majority (approximately 90%) of these malignancies involve neoplastic lesions in the squamous epithelial compartment of the mouth cavity, lip, and pharynx. In oral cancer patients, death usually occurs as a result of local invasion into the stromal tissue of head & neck and cervical lymph node metastases (Woolgar et al., Oral oncology. 2003 February; 39(2):130-7, Myers et al., Cancer. 2001 Dec. 15; 92(12):3030-6).

Decorin is a member of the small leucine-rich repeat proteoglycans (SLRPs) family and is primarily synthesized by fibroblasts and myofibroblasts (Hocking et al., Matrix Biol. 1998 April; 17(1):1-19). Members of the SLRPs family are structurally related and play major roles in the organization of the extracellular matrix (ECM) and the regulation of cell behaviour (Iozzo RV. The Journal of biological chemistry. 1999 July 2; 274(27):18843-6). SLRPs have a 40-50 kDa protein core with central leucine rich repeat (LRR) domains characterized by a common molecular architecture adapted for protein-protein interaction (Kobe et al., Current opinion in structural biology. 2001 December: 11(6):725-32). Decorin is normally present in the extracellular stromal compartment and has a prominent biological function in transforming growth factor (TGF)-beta and epidermal growth factor receptor activation pathways that contributes to its role in cellular proliferation, angiogenesis, and immunomodulation. Decorin is rarely expressed by cancer tissue as has been demonstrated by analysis of a variety of tumors including colon, pancreas, prostate, lung, ovarian, breast cancer (Iozzo and Cohen, Experientia. 1993 May 15; 49(5):447-55, McDoniels-Silvers et al., Clin Cancer Res. 2002 April; 8(4):1127-38, Shridhar et al., Cancer research. 2001 August. 1; 61(15):5895-904, Troup et al., Clin Cancer Res. 2003 January; 9(1):207-14). However, it is expressed in the tumour stroma and has been shown to inhibit tumour cells growth and trigger apoptosis (De Luca et al., The Journal of biological chemistry. 1996 August. 2; 271(31):18961-5, Nash et al., Cancer research. 1999 Dec. 15; 59(24):6192-6, Seidler et al., The Journal of biological chemistry. 2006 Sep. 8; 281(36):26408-18). On the contrary, it has been shown that decorin is produced by oral squamous cell carcinoma and osteosarcoma cells (Banerjee et al., Cancer research. 2003 November. 15; 63(22):7769-76, Zafiropoulos et al., Mol Cancer Res. 2008 May; 6(5):785-94). Osteosarcoma cells were reported not to be sensitive to decorin-induced growth arrest, rather decorin seemed to be beneficial, since it was necessary for osteosarcoma cell migration (Zafiropoulos et al., Connective tissue research. 2008; 49(3):244-8).

Toll-like receptors (TLR), mainly expressed by immune related cells and epithelial cells, have emerged as keys players in the detection of pathogens and the induction of anti-microbial immune response. TLR recognize pathogen associated molecular patterns and trigger antimicrobial innate immune responses, mainly pro-inflammatory mediators, and thus are known to regulate the adaptive immune responses. A total of 13 mammalian TLR have been described, 11 of which are expressed in humans (reviewed in O'Neill, Current opinion in immunology. 2006 February; 18(1):3-9). Recently TLR expression or up-regulation has been detected in various tumour types, especially in epithelium derived cancers (Furrie et al., Immunology. 2005 August; 115(4):565-74, Kelly et al., Cancer research. 2006 April. 1; 66(7):3859-68, Lee et al., Molecular carcinogenesis. 2007 November; 46(11):941-7). Expression of TLRs varies in different cancerous cell types; however, evidence indicates that TLR expression is functionally associated with tumorigenesis. It has been suggested that TLR expression may promote malignant transformation of epithelial cells (Lee et al., Molecular carcinogenesis. 2007 November; 46(11):941-7, Kim et al., Int J Gynecol Cancer. 2008 March-April; 18(2):300-5). Engagement of TLRs promotes tumour development and protects the cancerous cells from immune attack, and induces resistance to apoptosis and chemo-resistance in some malignancies (Kelly et al., Cancer research. 2006 April. 1; 66(7):3859-68, He et al., Molecular immunology. 2007 April; 44(11):2850-9, Droemann et al., Respiratory research. 2005; 6:1).

TLR5 is one of the major TLRs expressed in epithelial cells. It is a receptor for flagellin protein from gram-positive and gram-negative bacterial flagella (Smith et al., Current topics in microbiology and immunology. 2002; 270:93-108). Stimulation of TLR5 leads to production of proinflammatory cytokines and chemokines e.g., interleukin 8 (IL-8, also termed as CXCL8). TLR5 expression has been shown to be associated with tumor progression in various cancers (Kim et al., Int J Gynecol Cancer. 2008 March-April; 18(2):300-5, Schmausser et al., Int J Med. Microbiol. 2005 June; 295(3):179-85). IL-8 is known to promote carcinoma progression by its angiogenic potential as well as by a direct effect on tumour invasion and metastasis via corresponding chemokine receptors CXCR1 and CXCR2 (Kitadai et al., British journal of cancer. 1999 October; 81(4):647-53, Kitadai et al., Clin Cancer Res. 2000 July; 6(7):2735-40).

### SUMMARY OF THE INVENTION

Provided herein are methods for decreasing expression of a decorin polypeptide in a cell. The methods include contacting a cell, such as an oral epithelial cell, with an effective amount of a polynucleotide that includes a nucleotide sequence substantially identical to, or substantially complementary to, consecutive nucleotides of a target mRNA encoding a decorin polypeptide. The method further includes measuring the decorin polypeptide in the cell, where the cell with the polynucleotide has less decorin polypeptide when compared to decorin polypeptide present in a corresponding control cell that does not comprise the polynucleotide. The decorin polypeptide may be present in the nucleus and/or the cytoplasm. In some aspects, expression of the decorin polypeptide is undetectable.

The oral epithelial cell may be a dysplastic cell, a carcinoma cell, or a malignant cell. The oral epithelial cell may be ex vivo or in vivo. The oral epithelial cell may be a human cell. The polynucleotide may be double stranded, and may be present in a vector. It may include ribonucleotides and/or deoxynucleotides, or consist of either ribonucleotides or deoxynucleotides. The double stranded polynucleotide may be include a single strand that includes self-complementary portions, or it may include two separate complementary strands. A polynucleotide introduced into a cell may include one or more modifications, such as a modified nucleic acid sugar, a modified base, a modified backbone, or a combination thereof.

The double stranded polynucleotide may include a nucleotide sequence of between 19 and 29 nucleotides. In some aspects, the target mRNA is an A1 transcript variant or an A2 transcript variant. The polynucleotide may include a nucleotide sequence substantially identical to, or substantially complementary to, consecutive nucleotides in exon 1, exon 2, exon 3a, exon 4, exon 5, exon 6, exon 7, exon 8, or exon 9, or consecutive nucleotides spanning exons 1 and 2, exons 2 and 3a, exons 3a and 4, exons 4 and 5; or exons 5 and 6. In one non-limiting example, the polynucleotide includes at least 19 consecutive nucleotides selected from GAAGAACCTTCACGCATTGAT (SEQ ID NO:6), or the complement thereof.

The method of claim 1 may further include measuring the motility of the cell. Typically, a cell with decreased decorin expression also has decreased motility when compared to the control cell.

Also provided herein are double stranded polynucleotides, for instance, dsRNAs that inhibit expression of a polynucleotide encoding a decorin polypeptide. The double stranded polynucleotide may include a nucleotide sequence substantially identical to, or complementary to, consecutive nucleotides of exon 1, exon 2, exon 3a, exon 4, exon 5, exon 6, exon 7, exon 8, or exon 9, such as consecutive nucleotides of exon 1, exon 2, exon 3a, or exon 5, or consecutive nucleotides spanning exons 1 and 2, exons 2 and 3a, exons 3a and 4, exons 4 and 5, or exons 5 and 6.

Further provided herein are methods for identifying an agent that alters the distribution of decorin polypeptide in a cell. The method may include contacting an oral epithelial cell with an agent, incubating the oral epithelial cell and the agent under conditions suitable for growth of the oral epithelial cell, and measuring the decorin polypeptide present in the nucleus and/or cytoplasm of the oral epithelial cell, wherein the oral epithelial cell contacted with the agent having less decorin polypeptide present in the nucleus and/or cytoplasm when compared to decorin polypeptide present in the nucleus and/or cytoplasm of a corresponding control cell that does not include the agent indicates the agent alters the distribution of decorin polypeptide in a cell.

Provided herein are methods for determining a prognosis for oral cancer in a subject. The methods may include providing an oral epithelial cell from a subject, contacting the cell with a compound that binds decorin polypeptide, and detecting the presence of a decorin polypeptide in an oral epithelial cell, wherein the presence of the polypeptide associated with the nucleus and/or cytoplasm of the oral epithelial cell indicates a prognosis of increased risk of oral cancer, and the absence of the polypeptide associated with the nucleus or cytoplasm of the oral epithelial cell indicates a prognosis of decreased risk of oral cancer. The compound may be an antibody that specifically binds to a decorin polypeptide, such as an antibody that specifically binds to a decorin polypeptide encoded by an A1 transcript variant or an A2 transcript variant.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Validation of stable knockdown of decorin in DOK and SCC-25 cells. DOK and SCC-25 cells were stably transfected with decorin-shRNA (DCN-shRNA), or scrambled sequence-shRNA (Ctrl-shRNA) or no transfection control (WT). A, RNA was extracted and cDNA was subjected to quantitative RT-PCR, normalized decorin expression from one representative experiment of three. B, Nuclear lysates were extracted and were subjected to SDS-PAGE followed by immunoblotting with anti-decorin and anti-β-tubulin antibodies. Data presented is one representative immuoblot of at least three experiments. ***p<0.001 compared to respective controls.

FIG. 2. Decorin silencing does not affect DOK or SCC-25 cell growth/proliferation. WT, control, and decorin silenced DOK and SCC-25 cells were cultured for 24 h. During the last hour of culture, 20 μl of CellTiter 96® Aqueous One Solution Reagent containing a tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine ethosulfate; PES) was added to the media (100 μl per well), and color changes were recorded by absorbance at 490 nm. Data are presented as mean±SE of three replicates of one representative experiment of three.

FIG. 3. TLR5 expression down regulation in decorin silenced DOK and SCC25 cells. RNA was extracted from WT, control and decorin silenced DOK and SCC-25 cells and cDNA was subjected to A, multiplex PCR as described in materials and methods, B quantitative RT-PCR, normalized TLR5 expression from one representative experiment of three. C, Cell lysates were collected as described in materials and methods and subjected to SDS-PAGE followed by immunoblotting using anti-TLR5 and anti-β-tubulin antibodies. D, Densitometric analysis is presented as a histogram of TLR5 relative band density from 3 experiments. ***p<0.001 compared to respective controls.

FIG. 4. Reduced IL-8 production in decorin Silenced DOK and SCC25. RNA was extracted from WT, control, and decorin silenced DOK and SCC-25 cells and cDNA was subjected to A, multiplex PCR as described in materials and methods, B quantitative RT-PCR, normalized IL-8 expression from one representative experiment of three. C, Cells were cultured without; or with D, 100 ng/ml flagellin and IL-8 was measured in 24 hours culture supernatants using ELISA. Data are presented as mean±SD of three replicates of one representative experiment of four. ***p<0.001 compared to respective controls.

FIG. 5. Migration and invasion suppression in decorin silenced cell lines. A, Cell motility through uncoated filters (migration) was measured 22 hours after plating. The migrating cells were fixed, stained, and photographed as described in materials and methods. Each panel represents one representative field of five from duplicate filters of three experiments. B, Migrated cells in each one of the five fields of duplicate filters were counted, numbers represent mean±SD of three experiments. C, Cells that invaded across the Matrigel™ layer were fixed, stained, and photographed. Each panel represents one representative field of five from duplicate filters of three experiments. D, Migrated and invaded cells in five fields of duplicate filters were counted and % invasion was calculated as described in materials and methods. Numbers represent mean±SD of three individual experiments. **p<0.01, ***p<0.001 compared to respective controls.

FIG. 6. Nucleotide sequence of a genomic human decoin polynucleotide (Genebank accession number NG_{—}011672, SEQ ID NO:1). Exon 1, nucleotides 5001-5375; exon 2, nucleotides 8448-8668; exon 3a, nucleotides 9445-9688; exon 3b, nucleotides 9478-9688; exon 4, nucleotides 23313-23425; exon 5, nucleotides 29521-29734; exon 6, nucleotides 30842-30955; exon 7, nucleotides 34841-34934; exon 8, nucleotides 36238-36376; and exon 9, nucleotides 41778-42772.

FIG. 7. Nucleotide and amino acid sequences of transcript variants and decorin isoforms. A1 transcript variant (GenBank accession number NM_{—}001920) and amino acid sequence of decorin isoform A1 (SEQ ID NO:3 and SEQ ID NO:4, respectively), exon 1, nucleotides 1-375; exon 3a, nucleotides 376-619; exon 4, nucleotides 620-732; exon 5, nucleotides 733-946; exon 6, nucleotides 947-1060; exon 7, nucleotides 1061-1154; exon 8, nucleotides 1155-1293; and exon 9, nucleotides 1294-2288. A2 transcript variant (GenBank accession number NM_{—}133503) and amino acid sequence of decorin isoform A2 (SEQ ID NO:4 and SEQ ID NO:5, respectively), exon 2, nucleotides 1-221; exon 3a, nucleotides 222-465; exon 4, nucleotides 466-578; exon 5, nucleotides 579-792; exon 6, nucleotides 793-906; exon 7, nucleotides 907-1000; exon 8, nucleotides 1001-1139, and exon 9, nucleotides 1140-2134.

### DETAILED DESCRIPTION OF ILLUSTRATIVE EMBODIMENTS

The present invention includes polynucleotides and the uses thereof. As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. Preferably, a polynucleotide of the present invention is isolated. An “isolated” polynucleotide is one that has been removed from its natural environment. Polynucleotides that are produced by recombinant, enzymatic, or chemical techniques are considered to be isolated and purified by definition, since they were never present in a natural environment. As used herein, “coding region” and “coding sequence” are used interchangeably and refer to a nucleotide sequence that encodes an mRNA or an unprocessed preRNA (i.e., an RNA molecule that includes both exons and introns) that is processed to produce an mRNA. As used herein, a “target coding region” and “target coding sequence” refer to a specific coding region whose expression is inhibited by a polynucleotide of the present invention. As used herein, a “target mRNA” is an mRNA encoded by a target coding region. Unless noted otherwise, a target coding region can result in multiple mRNAs distinguished by the use of different combinations of exons. Such related mRNAs are referred to as splice variants or transcript variants of a coding region.

Polynucleotides of the present invention include, but are not limited to, double stranded RNA (dsRNA) polynucleotides. The sequence of a polynucleotide of the present invention includes one strand, referred to herein as the sense strand, of between 19 and 29 nucleotides, for instance, 19, 20, 21, 22, 23, 24, 25, 26, 27, 28, or 29 nucleotides. The sense strand is substantially identical, preferably, identical, to a target mRNA. As used herein, the term “identical” means the nucleotide sequence of the sense strand has the same nucleotide sequence as a portion of the target mRNA. As used herein, the term “substantially identical” means the sequence of the sense strand differs from the sequence of a target mRNA at 1, 2, 3, or 4, preferably, 1 or 2 nucleotides, and the remaining nucleotides are identical to the sequence of the mRNA. These 1 to 4 nucleotides of the sense strand are referred to as non-complementary nucleotides. When a polynucleotide of the present invention includes a sense strand that is substantially identical to a target mRNA, the non-complementary nucleotides can be located anywhere in the polynucleotide (Birmingham et al., Nat. Meth., 3:199-204 (2006); Pei and Tuschl, Nat. Meth., 3:670-676 (2006)).

The other strand of a dsRNA polynucleotide, referred to herein as the antisense strand, includes nucleotides that are complementary to the sense strand. The antisense strand may be between 19 and 29 nucleotides, for instance, 19, 20, 21, 22, 23, 24, 25, 26, 27, 28, or 29 nucleotides. In some aspects, the sense strand and the antisense strand of a double stranded polynucleotide, preferably, a dsRNA, have different lengths (Marchques et al., Nat. Biotech., 24:559-565 (2006)). The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide. The polynucleotides of the present invention also include the double stranded DNA polynucleotides that correspond to the dsRNA polynucleotides of the present invention. Also included in the present invention are the single stranded RNA polyncleotides and single stranded DNA polynucleotides corresponding to the sense strands and antisense strands disclosed herein. It should be understood that the sequences disclosed herein as DNA sequences can be converted from a DNA sequence to an RNA sequence by replacing each thymidine nucleotide with a uracil nucleotide.

A polynucleotide of the present invention may include overhangs on one or both strands of a double stranded polynucleotide. An overhang is one or more nucleotides present in one strand of a double stranded polynucleotide that are unpaired, i.e., they do not have a corresponding complementary nucleotide in the other strand of the double stranded polynucleotide. An overhang may be at the 3′ end of a sense strand, an antisense strand, or both sense and antisense strands. An overhang is typically 1, 2, or 3 nucleotides in length. A preferred overhang is at the 3′ terminus and has the sequence thymine-thymine (or uracil-uracil if it is an RNA). Without intending to be limiting, such an overhang may be used to increase the stability of a dsRNA. If an overhang is present, it is preferably not considered a non-complementary nucleotide when determining whether a sense strand is identical or substantially identical to a target mRNA.

The sense and antisense strands of a dsRNA polynucleotide of the present invention may also be covalently attached, for instance, by a spacer made up of nucleotides. Such a polynucleotide is often referred to in the art as a short hairpin RNA (shRNA). Upon base pairing of the sense and antisense strands, the spacer region typically forms a loop. The number of nucleotides making up the loop can vary, and loops between 3 and 23 nucleotides have been reported (Sui et al., Proc. Nat'l. Acad. Sci. USA, 99:5515-5520 (2002), and Jacque et al., Nature, 418:435-438 (2002)).

Polynucleotides of the present invention are biologically active. A biologically active polynucleotide causes the post-transcriptional inhibition of expression, also referred to as silencing, of a target coding region. Without intending to be limited by theory, after introduction into a cell a polynucleotide of the present invention will hybridize with a target mRNA and signal cellular endonucleases to cleave the target mRNA. The result is the inhibition of expression of the polypeptide encoded by the mRNA. Whether the expression of a target coding region is inhibited can be determined, for instance, by measuring a decrease in the amount of the target mRNA in the cell, measuring a decrease in the amount of polypeptide encoded by the mRNA, or by measuring a decrease in the activity of the polypeptide encoded by the mRNA. As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably.

Polynucleotides of the present invention may be modified. Such modifications can be useful to increase stability of the polynucleotide in certain environments. Modifications can include a nucleic acid sugar, base, or backbone, or any combination thereof. The modifications can be synthetic, naturally occurring, or non-naturally occurring. A polynucleotide of the present invention can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide-nucleic acids. Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-β-trifluoromethyl nucleotides, T-O-ethyl-trifluoromethoxy nucleotides, 2′-β-difluoromethoxy-ethoxy nucleotides, or 2′-deoxy nucleotides. Polynucletotides can be obtained commercially synthesized to include such modifications (for instance, Dharmacon Inc., Lafayette, Colo.).

In one aspect, the present invention includes polynucleotides that inhibit expression of a polypeptide encoded by a decorin (DCN) coding region. As used herein a DCN coding region refers to the genomic nucleotide sequence disclosed at Genbank accession number NG_{—}011672 (SEQ ID NO:1). Several splice variants of the DCN coding region are expressed, such as A1, A2, B, C, D, and E (GenBank accession numbers NM_{—}001920 and NM_{—}133503 to 133507, respectively), that encode isoforms of the polypeptide decorin. Transcripts A1 and A2 (SEQ ID NO:2 and 4, respectively) encode the same protein isoform but have alternate 5′-untranslated regions arising from differential promoter activity and alternate exon splicing (Danielson et al., 1993, Genomics, 15:146-160). Transcript variant A1 is made up of exons 1, 3a, 4, 5, 6, 7, 8, and 9, and transcript variant A2 is made up of exons 2, 3a, 4, 5, 6, 7, 8, and 9. Exons 1, 2, 3a, and 5 are not present in transcript variants B, C, D, or E.

In some aspects, polynucleotides that inhibit expression of a polypeptide encoded by a DCN coding region includes a sequence that is present in only an A1 and/or A2 transcript variant. Examples of such sequences include, for instance, those present in exon 1 of the DCN coding region (nucleotides 5001-5375 of SEQ ID NO:1), those present in exon 2 of the DCN coding region (nucleotides 8448-8668 of SEQ ID NO:1), those present in exon 3a of the DCN coding region (nucleotides 9445-9688 of SEQ ID NO:1), and those present in exon 5 of the DCN coding region (nucleotides 29521-29734 of SEQ ID NO:1). Polynucleotides that inhibit expression of a target mRNA encoding a DCN polypeptide can span two adjacent exons, such, for example, exons 1 and 3a, exons 2 and 3a, exons 3a and 4, exons 4 and 5, or exons 5 and 6.

In other aspects, a target mRNA includes sequences present in exon 4 of the DCN coding region (nucleotides 23313-23425 of SEQ ID NO:1), sequences present in exon 6 of the DCN coding region (nucleotides 30842-30955 of SEQ ID NO:1), sequences present in exon 7 of the DCN coding region (nucleotides 34841-34934 of SEQ ID NO:1), sequences present in exon 8 of the DCN coding region (nucleotides 36238-36376 of SEQ ID NO:1), and sequences present in exon 9 of the DCN coding region (nucleotides 41778-42772 of SEQ ID NO:1).

Polynucleotides of the present invention that will act to inhibit expression of a decorin polypeptide include polynucleotides with a sense strand that is substantially identical or identical to a region of SEQ ID NO:1 that includes, for instance, nucleotides present in exon 1, 2, 3a, 4, 5, 6, 7, 8, or 9 as described. Examples of such polynucleotides that will act to inhibit expression of a polypeptide encoded by a DCN coding region include 5′-GAAGAACCTTCACGCATTGAT (SEQ ID NO:6). Other polynucleotides useful in the methods disclosed herein may be easily designed using routine methods.

As used herein a “decorin polypeptide” refers to a polypeptide having a molecular weight of 49 to 51 kilodaltons (kDa) as determined by sodium dodecyl sulfate (SDS) polyacrylamide gel electrophoresis, and bound by an antibody that specifically binds to a human decorin polypeptide, such as a polypeptide encoded by the nucleotide sequence disclosed at SEQ ID NO:2 or 4 (SEQ ID NO:3 or 5, respectively). Such antibodies are commercially obtainable from, for instance, R & D Systems (Minneapolis, Minn.) and Abeam, Inc. (Cambrige, Mass.), or may be produced as described herein. As used herein, an antibody that can specifically bind a polypeptide is an antibody that interacts only with the epitope of the antigen that induced the synthesis of the antibody, or interacts with a structurally related epitope. An antibody that specifically binds to an epitope will, under the appropriate conditions, interact with the epitope even in the presence of a diversity of potential binding targets.

A polynucleotide of the present invention can be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. Examples of viral vectors include, for instance, adenoviral vectors, adeno-associated viral vectors, lentiviral vectors, retroviral vectors, and herpes virus vectors. A vector may result in integration into a cell's genomic DNA. Typically, a vector is capable of replication in a bacterial host, for instance E. coli. Preferably the vector is a plasmid. A polynucleotide of the present invention can be present in a vector as two separate complementary polynucleotides, each of which can be expressed to yield a sense and an antisense strand of the dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include mammalian cells, such as murine cells and human cells. Suitable prokaryotic cells include eubacteria, such as gram-negative organisms, for example, E. coli.

An expression vector optionally includes regulatory sequences operably linked to the polynucleotide of the present invention. Typically, the promoter results in the production of an RNA polynucleotide. Examples of such promoters include, but are not limited to, those that cause binding of an RNA polymerase III complex to initiate transcription of an operably linked polynucleotide of the present invention. Examples of such promoters include U6 and H1 promoters. Vectors may also include inducible or regulatable promoters for expression of a polynucleotide of the present invention in a particular tissue or intracellular environment. The polynucleotide of the present invention also typically includes a transcription terminator. Suitable transcription terminators are known in the art and include, for instance, a stretch of 5 consecutive thymidine nucleotides.

Polynucleotides of the present invention can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide of the present invention in a cell, and the polynucleotide may then be isolated from the cell.

The present invention is also directed to compositions including one or more polynucleotides of the present invention. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.

A composition may be prepared by methods well known in the art of pharmacy. In general, a composition can be formulated to be compatible with its intended route of administration. Administration may be systemic or local. In some aspects local administration may have advantages for site-specific, targeted disease management. Local therapies may provide high, clinically effective concentrations directly to the treatment site, without causing systemic side effects. Examples of routes of administration include parenteral, e.g., intravenous, intradermal, subcutaneous, oral, transdermal (topical), and transmucosal administration. Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a polynucleotide of the present invention) in the required amount in an appropriate solvent with one or a combination of ingredients enumerated above, as required, followed by filtered sterilization. Generally, dispersions are prepared by incorporating the active compound into a sterile vehicle, which contains a dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. An example of transdermal administration includes iontophoretic delivery to the dermis or to other relevant tissues.

The active compounds can also be administered by any method suitable for administration of polynucleotide agents, e.g., using gene guns, bio injectors, and skin patches as well as needle-free methods such as the micro-particle DNA vaccine technology disclosed by Johnston et al. (U.S. Pat. No. 6,194,389). Additionally, intranasal delivery is possible, as described in, for instance, Hamajima et al. Clin. Immunol. Immunopathol., 88, 205-210 (1998). Deliver reagents such as lipids, cationic lipids, phospholipids, liposomes, and microencapsulation may also be used.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art.

A polynucleotide described herein may be used in combination with other agents assisting the cellular uptake of polynucleotides, or assisting the release of polynucleotides from endosomes or intracellular compartments into the cytoplasm or cell nuclei by, for instance, conjugation of those to the polynucleotide. The agents may be, but are not limited to, peptides, especially cell penetrating peptides, protein transduction domains, and/or dsRNA-binding domains which enhance the cellular uptake of polynucleotides (Dowdy et al., US Published Patent Application 2009/0093026, Eguchi et al., 2009, Nature Biotechnology 27:567-571, Lindsay et al., 2002, Curr. Opin. Pharmacol., 2:587-594, Wadia and Dowdy, 2002, Curr. Opin. Biotechnol. 13:52-56. Gait, 2003, Cell. Mol. Life. Sci., 60:1-10). The conjugations can be performed at an internal position at the oligonucleotide or at a terminal postions either the 5′-end or the 3′-end.

Toxicity and therapeutic efficacy of such active compounds can be determined by standard pharmaceutical procedures in cell cultures or experimental animals, e.g., for determining the ED₅₀ (the dose therapeutically effective in 50% of the population).

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the methods of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of signs and/or symptoms) as determined in cell culture. Such information can be used to more accurately determine useful doses in humans.

The compositions can be administered one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of a polynucleotide can include a single treatment or can include a series of treatments.

The polynucleotides of the present invention can be designed using methods that are routine and known in the art. For instance, polynucleotides that inhibit the expression of a decorin polypeptide may be identified by the use of cell lines and/or primary cells. A candidate polynucleotide is the polynucleotide that is being tested to determine if it decreases expression of a decorin polypeptide described herein. The candidate polynucleotide can be identical to nucleotides located in the region encoding the polypeptide, or located in the 5′ or 3′ untranslated regions of the mRNA. Other methods are known in the art and used routinely for designing and selecting candidate polynucleotides. Candidate polynucleotides are typically screened using publicly available algorithms (e.g., BLAST) to compare the candidate polynucleotide sequences with coding sequences. Those that are likely to form a duplex with an mRNA expressed by a non-target coding region are typically eliminated from further consideration. The remaining candidate polynucleotides may then be tested to determine if they inhibit expression of one of the polypeptides described herein.

In general, candidate polynucleotides are individually tested by introducing a candidate polynucleotide into a cell that expresses the appropriate polypeptide. The candidate polynucleotides may be prepared in vitro and then introduced into a cell. Methods for in vitro synthesis include, for instance, chemical synthesis with a conventional DNA/RNA synthesizer. Commercial suppliers of synthetic polynucleotides and reagents for such synthesis are well known. Methods for in vitro synthesis also include, for instance, in vitro transcription using a circular or linear vector in a cell free system.

The candidate polynucleotides may also be prepared by introducing into a cell a construct that encodes the candidate polynucleotide. Such constructs are known in the art and include, for example, a vector encoding and expressing a sense strand and an antisense strand of a candidate polynucleotide, and RNA expression vectors that include the sequence encoding the sense strand and an antisense strand of a candidate polynucleotide flanked by operably linked regulatory sequences, such as an RNA polymerase III promoter and an RNA polymerase III terminator, that result in the production of an RNA polynucleotide.

A cell that can be used to evaluate a candidate polynucleotide may be a cell that expresses the appropriate polypeptide. A cell can be ex vivo or in vivo. As used herein, the term “ex vivo” refers to a cell that has been removed from the body of a subject. Ex vivo cells include, for instance, primary cells (e.g., cells that have recently been removed from a subject and are capable of limited growth in tissue culture medium), and cultured cells (e.g., cells that are capable of extended culture in tissue culture medium). As used herein, the term “in vivo” refers to a cell that is within the body of a subject. Whether a cell expresses one of the polypeptides can be determined using methods that are routine and known in the art including, for instance, Western immunoblot, ELISA, immunoprecipitation, or immunohistochemistry. Western immunoblot and immunoprecipitation are generally used with ex vivo cells, and immunohistochemistry is generally used with in vivo cells. Examples of readily available cells expressing a polypeptide encoded by a DCN coding region include cultured cells such as, but not limited to, HOK16B, SCC4, SCC25, SCC66, DOK, and OSC-2 cell lines, and primary cells obtained from biopsy, such as cells present in a precancerous or cancerous lesion in a tissue of epithelial origin from a subject's head and/or neck, such as mouth cavity, lip, nasal cavity, paranasal sinuses, pharynx, or larynx, or lymph nodes draining such tissues. Other cells can also be modified to express one of the polypeptides by introducing into a cell a vector having a polynucleotide encoding the polypeptide.

Candidate polynucleotides may also be tested in animal models. The study of various cancers in animal models (for instance, mice) is a commonly accepted practice for the study of cancers. For instance, the nude mouse model, where human tumor cells are injected into the animal, is commonly accepted as a general model useful for the study of a wide variety of cancers. Another animal model commonly accepted for the study of human oral cancers is spontaneously developing oral cancer in domesticated dogs. Candidate polynucleotides can be used in this and other animal models to determine if a candidate polynucleotide decreases one or more symptoms associated with the disease.

Methods for introducing a candidate polynucleotide into a cell, including a vector encoding a candidate polynucleotide, are known in the art and routine. When the cells are ex vivo, such methods include, for instance, transfection with a delivery reagent, such as lipid or amine based reagents, including cationic liposomes or polymeric DNA-binding cations (such as poly-L-lysine and polyethyleneimine). Alternatively, electroporation or viral transfection can be used to introduce a candidate polynucleotide, or a vector encoding a candidate polynucleotide. When the cells are in vivo, such methods include, but are not limited to, local or intravenous administration.

When evaluating whether a candidate polynucleotide functions to inhibit expression of one of the polypeptides described herein, the amount of target mRNA in a cell containing a candidate polynucleotide can be measured and compared to the same type of cell that does not contain the candidate polynucleotide. Methods for measuring mRNA levels in a cell are known in the art and routine. Such methods include quantitative reverse-transcriptase polymerase chain reaction (RT-PCR). Primers and specific conditions for amplification of an mRNA encoding a DCN polypeptide can be readily determined by the skilled person. An example of useful primers for RT-PCR includes GGACCGTTTCAACAGAGAGG (SEQ ID NO:7) and GACCACTCGAAGATGGCATT (SEQ ID NO:8). Other methods include, for instance, Northern blotting, and array analysis.

Other methods for evaluating whether a candidate polynucleotide functions to inhibit expression of one of the polypeptides described herein include monitoring the polypeptide. For instance, assays can be used to measure a decrease in the amount of polypeptide encoded by the mRNA, or to measure a decrease in the activity of the polypeptide encoded by the mRNA. Methods for measuring a decrease in the amount of a polypeptide include assaying for the polypeptide present in cells containing a candidate polynucleotide and comparing to the same type of cell that does not contain the candidate polynucleotide. For instance, antibody specific for the polypeptides described herein can be used in Western immunoblot, immunoprecipitation, or immunohistochemistry.

A candidate polynucleotide that is able to decrease the expression of a polypeptide encoded by a DCN coding region by at least 50%, at least 60%, at least 70%, at least 80%, or at least 90% when compared to a control cell, is considered to be a polynucleotide of the present invention.

The present invention is further directed to methods of using the polynucleotides described herein. dsRNA described herein mediate RNA interference (RNAi) of a target mRNA. RNAi is the process of sequence-specific, post-transcriptional gene silencing in animals and plants, initiated by double-stranded RNA (dsRNA) that is identical or substantially identical in sequence to the silenced gene. Methods relating to the use of RNAi to silence expression of a target coding sequence are known to the person skilled in the art. Methods of the present invention include decreasing the amount of decorin polypeptide in a cell, decreasing cell migration, decreasing cell invasion, decreasing expression of Toll-like receptor TLR5 in a cell, and/or decreasing IL-8 expression in a cell. Methods for measuring changes in decorin polypeptide, TLR5 expression, IL-8 expression, cell migration and/or cell invasion are known in the art and routine. Typically, the presence of one of these characteristics, such as decorin polypeptide, of a cell can be compared with the same type of cell that does not contain the polynucleotide of the invention. Such a cell that does not contain the polynucleotide is referred to as a control cell. A decrease in, for instance, the target mRNA or the amount of polypeptide encoded by the target mRNA in the cell containing a polynucleotide of the present invention indicates the expression of the polypeptide has been inhibited.

In some aspects methods of the present invention include treating certain diseases in a subject in need of treatment. The subject is a mammal, including members of the family Muridae (a murine animal such as rat or mouse), a canine, such as a domesticated dog, and human, preferably a human. As used herein, the term “disease” refers to any deviation from or interruption of the normal structure or function of a part, organ, or system, or combination thereof, of a subject that is manifested by a characteristic sign or set of signs. As used herein, the term “sign” refers to objective evidence of a disease present in a subject. Signs associated with diseases referred to herein and the evaluation of such signs are routine and known in the art. Diseases include head and neck cancers. Such cancers are typically primary cancers, and can include cancerous cells that are not metastatic, and cancerous cells that are metastatic. Examples of such cancers are squamous cell carcinomas and adenocarcinomas, such as oral cancer, nasopharyngeal cancer, oropharyngeal squamous cell carcinoma, cancer of the hypopharynx, laryngeal cancer, and cancer of the trachea. Other diseases can include cancers resulting from metastasis of a cancer, such as metastasis of a primary cancer. The metastatic cancer can be located in, for instance, the lymph nodes of the neck. Typically, whether a subject has a disease, and whether a subject is responding to treatment, may be determined by evaluation of signs associated with the disease.

Treatment of a disease can be prophylactic or, alternatively, can be initiated after the development of a disease. Treatment that is prophylactic, for instance, initiated before a subject manifests signs of a disease, is referred to herein as treatment of a subject that is “at risk” of developing a disease. An example of a subject that is at risk of developing a disease is a person having a risk factor, such as alcohol and/or tobacco use, dietary factors, UV light and occupational exposures, and certain strains of viruses, such as the sexually transmitted human papillomavirus. Treatment can be performed before, during, or after the occurrence of the diseases described herein. Treatment initiated after the development of a disease may result in decreasing the severity of the signs of the disease, or completely removing the signs.

In some aspects, the methods typically include contacting under conditions suitable for introduction into the cell an effective amount of one or more polynucleotides of the present invention. Conditions that are “suitable” for an event to occur, such as introduction of a polynucleotide into a cell, or “suitable” conditions are conditions that do not prevent such events from occurring. Thus, these conditions permit, enhance, facilitate, and/or are conducive to the event. As used herein, an “effective amount” is an amount effective to inhibit expression of a decorin polypeptide in a cell, decrease signs associated with a disease, or the combination thereof. The polynucleotide may be introduced into a cell as a dsRNA polynucleotide, or as a vector including a DNA polynucleotide that encodes and will express the RNA polynucleotide. More than one type of polynucleotide can be administered. For instance, two or more polynucleotides that are designed to silence the same mRNA can be combined and used in the methods herein. Whether a polynucleotide is expected to function in methods of the present invention relating to treatment can be evaluated using ex vivo models and animal models. Such models are known in the art and are generally accepted as representative of disease in humans and useful for evaluation of methods of treating humans.

The cells may be in vivo or ex vivo. The cells may be of epithelial origin, such as epithelial cells present in the head and/or neck of an animal, for instance, epithelial cells in the mouth cavity, lip, nasal cavity, paranasal sinuses, pharynx, or larynx. Epithelial cells from the head and/or neck of a subject including mouth cavity, lip, nasal cavity, paranasal sinuses, pharynx, or larynx, are referred to herein as oral epithelial cells. The cells are animal cells, such as vertebrate cells, including murine (rat or mouse), canine, or primate cells, such as human cells. The cells may be dysplastic cells, carcinoma cells, or malignant cells. Ex vivo and in vivo cells may be obtained from or present in, respectively, pre-cancerous or cancerous lesions in a subject.

The methods of the present invention can include administering to a subject having a disease or at risk of developing a disease a composition including an effective amount of a polynucleotide of the present invention, wherein expression of a polypeptide in a cell is decreased, a sign associated with the disease is decreased, or a combination thereof. Preferred methods for administering one or more of the polynucleotides of the present invention include administration during surgery, for instance surgery to resect a diseased part, organ, system, or combination thereof, of a subject. A diseased part, organ, or system can include, for instance, tumor cells. For instance, after removal of cancer cells the surrounding area can be perfused with a solution containing one or more of the polynucleotides of the present invention, or an implant containing one or more of the polynucleotides of the present invention can be placed near the area of resection. The polynucleotides may also be administered by other methods known in the art including, for instance, intravenous administration.

The polynucleotides of the present invention can also be administered to a subject in combination with other therapeutic compounds to increase the overall therapeutic effect. Therapeutic compounds useful for the treatment of the diseases described herein are known and used routinely. A wide variety of antitumor agents are available that may be used as a second, supplemental agent, to complement the activity of the polynucleotides described herein. Antitumor agents that have proven particularly effective in treating head and neck cancers include, for instance, monoclonal antibodies to EGFR receptors (Cituximab™).

The present invention provides methods for detecting decorin polypeptide in a cell. Decorin polypeptide is typically produced and transported out of cells, and is not typically present in cells. Decorin polypeptide has been shown to be aberrantly expressed as well as translocated to the nucleus in dysplastic oral keratinocytes and malignant squamous cell carcinoma and in oral cancer biopsy tissue (Banerjee et al., 2003, Cancer Res., 63: 7769-7776). As described in Example 1, the presence of decorin polypeptide in the nucleus or cytoplasm of an oral epithelial cell obtained from a subject indicates the subject is at risk of developing, or has, regional metastases of a primary lesion. Thus, methods of the present invention also include determining a prognosis for oral cancer in a subject. The methods typically include providing an oral epithelial cell from a subject, and detecting the presence of a decorin polypeptide in an oral epithelial cell. The presence of the polypeptide associated with the nucleus or cytoplasm of the oral epithelial cell indicates a prognosis of increased risk of oral cancer and/or regional metastases, and the absence of the polypeptide associated with the nucleus or cytoplasm of the oral epithelial cell indicates a prognosis of decreased risk of oral cancer and/or regional metastases.

The oral epithelial cell may be obtained by biopsy of tissue suspected of including a lesion with dysplastic, carcinoma, or malignant cells. The biopsy may be from, for instance, a subject's head and/or neck, such as mouth cavity, lip, nasal cavity, paranasal sinuses, pharynx, or larynx, or lymph nodes draining such tissues. The cells may then be processed with routine methods known in the art. Such processing may include embedding in paraffin, and fixing thin sections to slides for further analysis.

Decorin polypeptide can be detected using an antibody or other compound that specifically binds to a decorin polypeptide. The decorin polypeptide detected may be isoform A1, A2, B, C, D, or E, preferably A1 or A2. In some aspects, the antibody or other compound specifically binds to a polypeptide corresponding to a particular exon of a DCN coding region. For instance, specific detection of a decorin polypeptide isoform encoded by an A1 or A2 transcript variant may be accomplished by use of an antibody or compound that specifically binds a polypeptide encoded by an exon present in an A1 or A2 transcript variant, such as exon 1, 2, 3a, or 5. The present invention also includes antibody that specifically binds to a polypeptide encoded by an exon present in an A1 or A2 transcript variant, such as exon 1, 2, 3a, or 5 of a DCN coding region, such as the DCN coding region depicted at SEQ ID NO:1.

Preferably, an antibody or other specific binding compound includes a label. As used herein, the tem' “label” refers to a compound that permits the detection of the antibody. Typically, when an antibody includes a label, the label is covalently attached to the antibody. Examples of such compounds include, for instance, fluorescent compounds (e.g., green, yellow, blue, orange, or red fluorescent proteins and non-proteins), aminomethylcoumarin, fluorescein, luciferase, alkaline phosphatase, and chloramphenicol acetyl transferase, and other molecules detectable by their fluorescence or enzymatic activity. Other examples of such compounds include biotin and other compounds that permit the use of a secondary compound that includes a detectable compound. Methods for the covalent attachment of label to an antibody or other specific binding compounds are routine and known to those skilled in the art. Attachment may be conducted by one skilled in the art, or antibodies conjugated to label may be obtained commercially from a suitable company (e.g. Molecular Probes, ALT, Quantum Dot)

“Antibody,” as used herein, includes human, non-human, or chimeric immunoglobulin, or binding fragments thereof, that specifically bind to an antigen.

Suitable antibodies may be polyclonal, monoclonal, or recombinant, or useful fragments such as Fab. Methods of preparing, manipulating, labeling, and using antibodies are well known in the art. See, e.g., Current Protocols In Molecular Biology, Greene Publishing and Wiley-Interscience, edited by Ausubel et al., including Supplement 46 (April 1999). Antibody that specifically binds to a polypeptide encoded by an exon present in an A1 or A2 transcript variant, such as exon 1, 2, 3a, or 5 (nucleotides 5001-5375, nucleotides 8448-8668, nucleotides 9445-9688, and nucleotides 29521-29734, respectively) may be produced using such polypeptides, or fragments thereof. Many suitable antibodies are also available commercially.

The present invention also includes methods for identifying an agent that alters the distribution of decorin polypeptide in a cell. The method includes contacting a cell, such as an oral epithelial cell, with an agent, incubating the cell and the agent under conditions suitable for culturing the cell, and measuring the decorin poylpeptide present in the cell. The decorin polypeptide may be in the cytoplasm of the cell and/or in the nucleus of the cell. The cell contacted with the agent having less decorin polypeptide present when compared to decorin polypeptide present in a corresponding control cell that does not include the agent indicates the agent alters the distribution of decorin polypeptide in a cell. The agent can be a chemical compound, including, for instance, an organic compound, an inorganic compound, a metal, a polypeptide, a non-ribosomal polypeptide, a polyketide, or a peptidomimetic compound. The sources for potential agents to be screened include, for instance, chemical compound libraries, cell extracts of plants and other vegetations.

The present invention also provides kits for practicing the methods described herein. A kit includes one or more of the polynucleotides or antibodies of the present invention in a suitable packaging material in an amount sufficient for at least one use. Optionally, other reagents such as buffers and solutions needed to practice the invention are also included. Instructions for use of the packaged polynucleotide(s) or antibodies are also typically included.

As used herein, the phrase “packaging material” refers to one or more physical structures used to house the contents of the kit. The packaging material is constructed by well known methods, preferably to provide a sterile, contaminant-free environment. The packaging material has a label which indicates that the polynucleotide(s) or antibodies can be used for the methods described herein. In addition, the packaging material contains instructions indicating how the materials within the kit are employed to practice the methods. As used herein, the term “package” refers to a solid matrix or material such as glass, plastic, paper, foil, and the like, capable of holding within fixed limits the polynucleotide(s) or antibodies. Thus, for example, a package can be a glass vial used to contain appropriate quantities of the polynucleotide(s) or antibodies. “Instructions for use” typically include a tangible expression describing the conditions for use of the polynucleotide(s) or antibodies.

The present invention is illustrated by the following examples. It is to be understood that the particular examples, materials, amounts, and procedures are to be interpreted broadly in accordance with the scope and spirit of the invention as set forth herein.

### Examples

The function of nuclear decorin in oral cancer progression was examined using a post-transcriptional gene silencing approach in DOK and SCC-25 cells. More than 80% decorin silencing was achieved as confirmed by real time PCR and western blot analysis. Decorin knock down caused significant down regulation of Toll-like receptor 5 (TLR5) in both cell types and was consequently accompanied by significant reduction in IL-8 production in both DOK and SCC-25 cells, even after flagellin stimulation. Silencing of decorin expression did not alter cell proliferation in either cell type, however; invasive and migratory phenotype of DOK and SCC-25 cells was found to be significantly reduced as measured by Matrigel™ coated and uncoated Trans well chamber assays respectively. Effect on abrogation of cellular invasion was more pronounced in DOK than in SCC-25 cells. Taken together, our results provide the first evidence that nuclear localized decorin plays an important role in oral cancer progression and is required for migration and invasion of dysplastic as well as malignant oral epithelial cells.

### Materials and Methods

Cell Lines. Oral epithelial origin, premalignant—Dysplastic Oral Keratinocyte (DOK) and malignant—Squamous Carcinoma Cell (SCC-25) lines were routinely maintained in DMEM/F 12 (Hyclone, Logan, Utah) supplemented with 10% Foetal Calf Serum for use as in vitro model in our studies, as described previously (Hu et al., Cancer research. 1991 August. 1; 51(15):3972-81, Hsu et al., Cell proliferation. 2002 June; 35(3):183-92).

Decorin knock down in DOK and SCC-25 cells in vitro. Silencing of decorin gene expression was achieved using short hairpin RNA (shRNA) technology. Oligonucleotides targeting decorin transcript variants-A1 (RefSeq accession no NM_{—}001920.3, at nucleotide position 720-740) and -A2 (RefSeq accession no NM_{—}133503.2, at nucleotide position 566-586) (GAAGAACCTTCACGCATTGAT, SEQ ID NO:6) and the corresponding scrambled sequence nonspecific to any gene were custom synthesized, annealed, and cloned into the shRNA expression vector pGeneClip Puro™ (Promega) by Super Array Bioscience Corporation (Frederick, Md.). BLAST queries were performed to ensure that the sequences have no significant homology with any other human genes. The transformation grade shRNAi plasmids were amplified in E. coli cultures, purified using Midiprep kits for endotoxin-free DNA vectors and then stably transfected into DOK and SCC-25 cells using Effectene™ transfection reagent following manufacturer's protocol (Qiagen, Valencia, Calif.). The positive transfectants were selected for puromycin (Calbiochem, San Diego, Calif.) antibiotic resistance at 2.5 μg/ml final optimal concentration. To avoid clone-specific variances, pools of stable transfectants (maintained at 1 μg/ml of puromycin) were used in all subsequent experiments. Decorin expression levels were determined at transcript and protein level by quantitative real-time reverse transcription-PCR(RT-PCR) and Western blotting, respectively. Hereafter, untransfected DOK and SCC-25 cells will be referred to as wild type (WT), scrambled shRNA stable transfectants as control (or Ctrl-shRNA in figures), and decorin shRNA stable transfectants as decorin silenced (or DCN-shRNA in figures).

Real-time PCR. RNA was extracted from DOK and SCC-25 cells using RNeasy Plus mini kit (Qiagen, Valencia, Calif.). Initially 2.5 μg of total RNA was used to synthesize cDNA, using SuperScript III Reverse Transcriptase (Invitrogen, San Diego, Calif.). Quantitative RT-PCR was performed using QuantiTect™ SYBR Green PCR kit (Qiagen, Valencia, Calif.) on the Mini Opticon™ Real-Time PCR system (BioRad, Hercules, Calif.) as per manufacturer's protocol. Quantitative PCR primer pairs were designed for SYBR Green chemistry based detection of amplicons for DCN (5′-GGACCGTTTCAACAGAGAGG (SEQ ID NO:17), 5′-GACCACTCGAAGATGGCATT (SEQ ID NO:18)), TLR5 (5′-TGCATTAAGGGGACTAAGCCT (SEQ ID NO:19), 5′-AAAAGGGAGAACTTTAGGGACT (SEQ ID NO:20)), IL-8 (5′-TCTGCAGCTCTGTGTGAAGG (SEQ ID NO:21), 5′-TAATTTCTGTGTTGGCGCAG-(SEQ ID NO:22)), and GAPDH (5′-ACAGTCAGCCGCATCTTCTT-(SEQ ID NO:23), 5′-GTTAAAAGCAGCCCTGGTGA (SEQ ID NO:24)). GAPDH was used as relative house-keeping gene expression control to normalized for sample variations.

Multiplex PCR. The transcript expression levels of innate immune receptors, co-regulatory molecules and cytokines were quantified in decorin silenced, control, and WT DOK and SCC25 cells using multiplex PCR (MPCR) kits for human signaling receptor set-2 (TLR1, TLR2, TLR3, TLR4, TLR5, TLR6, TLR 9 and CD14) and human Th1/Th2 cytokines set-4 (IL-2, IL-5, IL-8, IL-10, IL-14, TNF-α; and TGF-β1) from Maxim Biotech, San Francisco, Calif.) respectively. Both sets also included housekeeping gene -GAPDH, as internal cDNA loading control in each reaction. MPCR was carried out according to the manufacturer's instructions. Briefly, 1×MPCR buffer, 2.5 units of Taq DNA polymerase, and cDNA template from DOK and SCC25 cells were mixed in a 25 μl reaction and subjected to 35 cycles of PCR, with denaturing, annealing, and extension temperatures at 96, 67, and 70° C., respectively, for TLRs and 96, 60, and 70° C., respectively, for cytokines. Following MPCR, the DNA amplicons were fractionated electrophoretically on 2% agarose gel containing 0.5 μg/ml ethidium bromide.

Cell proliferation assay. Cell proliferation was measured using CellTiter 96®Aqueous One Solution -Cell Proliferation assay, which is an MTS based assay (Promega, Madison, Wis.) according to manufacturer's instructions. Briefly, WT, control and decorin silenced DOK and SCC-25 cells (10⁵ cells/well), were cultured in 96-well flat-bottom plates at a final volume of a 100 μl for 24, 48, and 72 h. During the last hour of culture 20 μl of CellTiter 96® Aqueous One Solution reagent, containing a tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine ethosulfate; PES), was added to each well. Increase in absorbance at 490 nm wavelength (indicating cell proliferation) was measured using a 96-well plate reader (SPECTRAMax 190, Molecular Devices, Sunnyvale, Calif.) and results were analyzed by SOFTMax Pro software. Western Blot Analysis. Cells were rinsed with ice-cold PBS and were lysed in a buffer containing 20 mM Tris, pH 7.6, 0.1% SDS, 1% Triton-X, 1% deoxycholate, 100 μg/ml PMSF, and protease inhibitor cocktail (Sigma-Aldrich, St. Louis, Mo.). Lysates were centrifuged at 20,000×g for 20 min at 4° C. Nuclear extracts were prepared by using NE-PER kit reagents (Pierce, Rockford, Ill.) following manufacturer's protocol. Protein concentration was determined by Bis-Cinchonic Acid (BCA) protein assay (Pierce, Rockford, Ill.) and subjected to 10% SDS-PAGE analysis, followed by transfer to polyvinylidene difluoride membrane (Bio-Rad, Hercules, Calif.). The membranes were immunoprobed with 1:500 dilution of monoclonal anti-human decorin antibody (Abeam, Cambridge, Mass.) or 1:500 dilution of monoclonal antibody to human TLR5 (Alexis Biochemicals, San Diego, Calif.) or 1:1000 dilution of anti-human β-tubulin polyclonal antibody. Western blots were developed with appropriate horseradish peroxidase conjugated secondary antibodies (Bio-Rad) and ECL Plus chemiluminescence system (Amersham, Arlington Heights, Ill.) and exposed to auto radiographic films. Radiographs were scanned and densitometry analysis was done using AlphaEase FC software (Alpha Innotech Corporation, San Leandro, Calif.).

ELISA for IL-8 Quantification. Decorin silenced, control and WT DOK and SCC-25 cells (5×10⁵ cells/well) were cultured in complete medium in 24-well flat-bottom plates at a final volume of a 500 μl. Cells were stimulated with varying concentrations of flagellin (Alexis Biochemicals, San Diego, Calif.); 100 ng/ml concentration was found to be optimal. Culture supernatants were collected after 24 h, 48 h or 72 h of incubation and IL-8 was assayed by ELISA. DuoSet IL-8 ELISA kit was purchased from R&D Systems (Minneapolis, Minn.), and ELISA was performed according to manufacturer's instructions with 100 μl of cell free culture supernatant. IL-8 detection limit was found to be 5.6 pg/ml. Absorbance was read at 450 nm with the SPECTRAMax 190 microplate spectrophotometer and results were analyzed by SOFTMax Pro software (Molecular Devices, Sunnyvale, Calif.). Sample concentrations were determined by interpolation from the standard curve. Samples were read in triplicate.

Cell Migration and Invasion Assay. The ability of cells to migrate across control inserts (migration) or invade across Matrigel™— coated inserts (invasion) was assayed using BD Falcon control inserts or BD BioCoat Matrigel™ invasion chambers (BD Biosciences, San Jose, Calif.), respectively. The BD BioCoat Matrigel™ invasion chambers consist of BD Falcon tissue culture companion plate with Falcon cell culture inserts containing 8 micron pore size PET membrane, pre-coated with a thin layer of Matrigel™ basement membrane matrix. Manufacturer's instructions were followed to perform the assay. Briefly, serum free DMEM/F12 medium (0.5 ml) containing 10⁵ cells were added to the upper chamber, and 0.75 ml of DMEM/F12medium containing 10% serum was added to the lower chamber as a chemo-attractant. After overnight incubation at 37° C. and 5% CO2, cells on the upper surface of the filter (cells that had not penetrated the filter) were removed using a cotton swab. Cells that had migrated to the lower surface of the filter were fixed in 100% methanol and stained with 0.005% crystal violet. For each filter, the number of migrated cells in 5 medium-power fields (magnification of 20×) was counted using bright field microscopy, and photographed. Assays were performed in duplicates and repeated at least three times. Invasion index is expressed as percentage of invading cells, and is calculated by dividing mean number of cells invading through Matrigel™ membrane over mean number of cells migrating through the non-coated control insert membrane per microscopic filed over five fields per assay, and ratio then multiplied by 100 for percent values.

Statistical Analysis. Student's paired t test was used to determine the statistical significance of the data. Statistical analysis was performed on Graph Pad Prism Software. Significance was evaluated at op values:

*p<0.05,**p<0.01,***p<0.001.

### Results

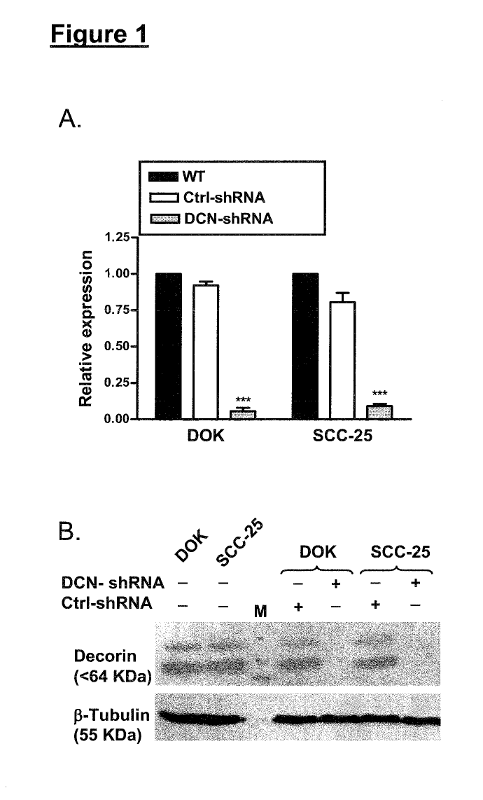

Stable knock down of decorin using shRNA in DOK and SCC-25. To study the functional role of aberrantly expressed nuclear decorin in dysplastic and malignant epithelial cells, decorin shRNA-stable clones were generated. Briefly, the DNA oligonucleotides specific for decorin and a scrambled control were generated and ligated into pGeneClip™ Puro plasmid, referred to as decorin shRNA (DCN-shRNA) and control shRNA (Ctrl-shRNA), respectively. DOK and SCC-25 cells were transfected with these constructs and puromycin resistant positive clones were selected. To avoid clone-specific effects, pooled transfectants were used for each cell type. Knock down of decorin expression was confirmed by real-time PCR and western blot analysis. Pooled decorin-shRNA transfected DOK clones showed a significant (more than 80%) decrease in decorin mRNA expression when compared to control-shRNA transfected clones or no transfection wild type DOK (FIG. 1A). Similar results were observed in SCC-25 cells (FIG. 1A). Decorin knock down was also confirmed by western blot. Pooled decorin-shRNA transfected DOK or SCC-25 clones showed almost complete abrogation of decorin protein expression in nuclear lysates (FIG. 1B). Similar decorin protein expression knock down was observed in whole cell lysates (data not shown). These results demonstrate that decorin-shRNA successfully silenced the nuclear decorin expression in DOK and SCC-25 cells.

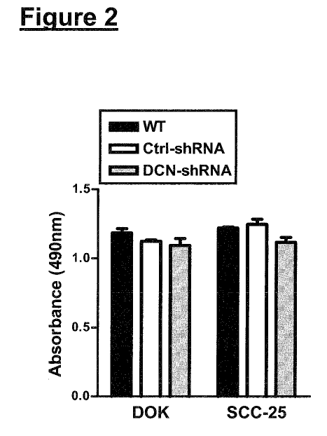

Decorin knock down does not affect cell proliferation in dysplastic and malignant epithelia. To evaluate the role of aberrantly expressed nuclear decorin on the cellular proliferation rates of dysplastic and malignant oral epithelial cells, DOK and SCC-25 WT cells, DCN-shRNA transfectants and ctrl-shRNA transfectants were allowed to grow in culture for 24, 48 and 72 h and proliferation was assessed by MTS assay. Compared with WT or control-shRNA cells, decorin silenced DOK and SCC-25 cells did not show any change in cell proliferation rates at 24 hrs (FIG. 2). Similar results were obtained at 48 and 72 h time points.

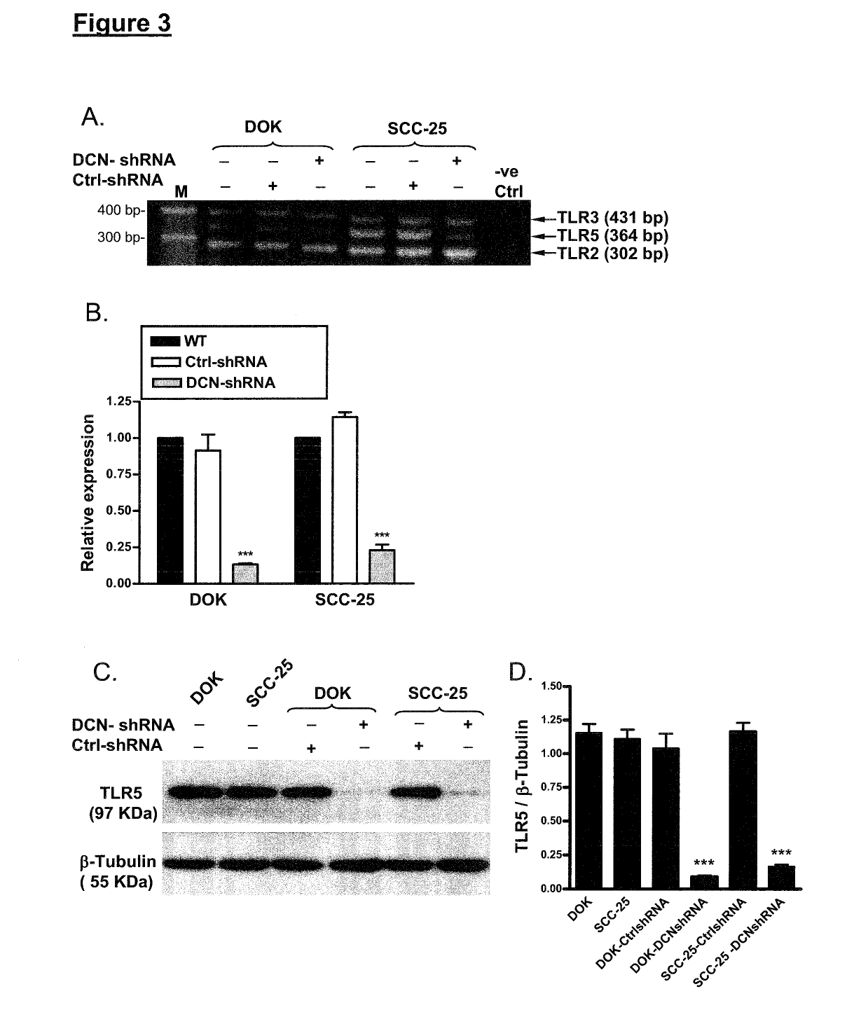

TLR5 expression down regulation in decorin silenced DOK and SCC25 cells. Toll-like receptor expression has been described in many cancers especially epithelial derived tumours and has been linked to tumour progression (Yu et al., Cancer Immunol Immunother. 2008 September; 57(9):1271-8). We sought to determine whether nuclear decorin silencing has an effect on any or all of the TLRs expression in dysplastic and malignant oral epithelial cells. Multiplex PCR analysis showed that out of a set of TLRs, TLR5 was significantly reduced in decorin silenced DOK and SCC-25 cells compared to respective WT and control cells (FIG. 3A). Interestingly, TLR2 and TLR3 were evenly expressed among WT, control and decorin silenced cells in either DOK or SCC-25 (FIG. 3A) and no difference was observed in the expression of TLR1 and TLR6 between decorin silenced and unsilenced cells (data not shown). Real time PCR analysis using TLR5 specific primers revealed more than 75% reduction in TLR5 expression in decorin silenced DOK and SCC-25 cells (FIG. 3B). Western blot analysis showed similar TLR5 protein reduction in decorin silenced DOK and SCC-25 cells in comparison to TLR5 expression in respective WT and/or control cells. It is interesting to note that malignant SCC-25 cells have a slightly higher expression of TLR5 than the dysplastic DOK cells.

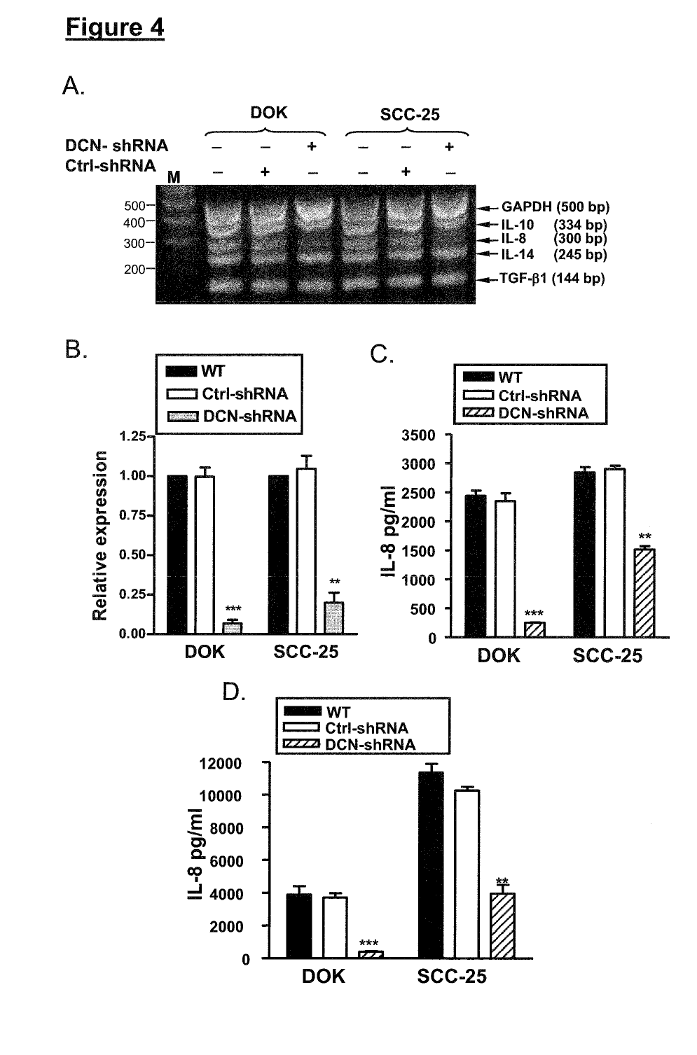

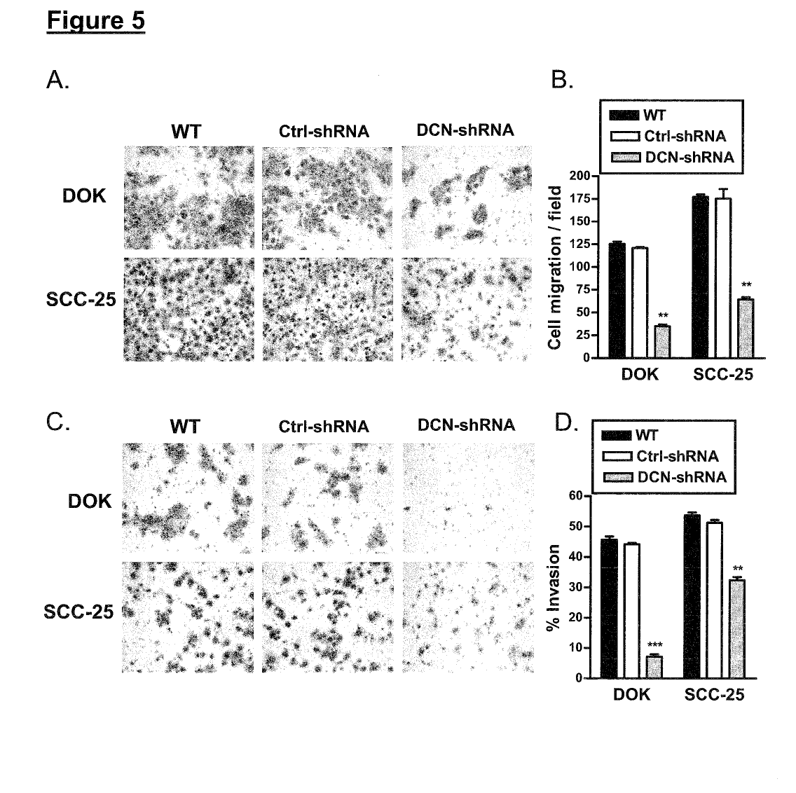

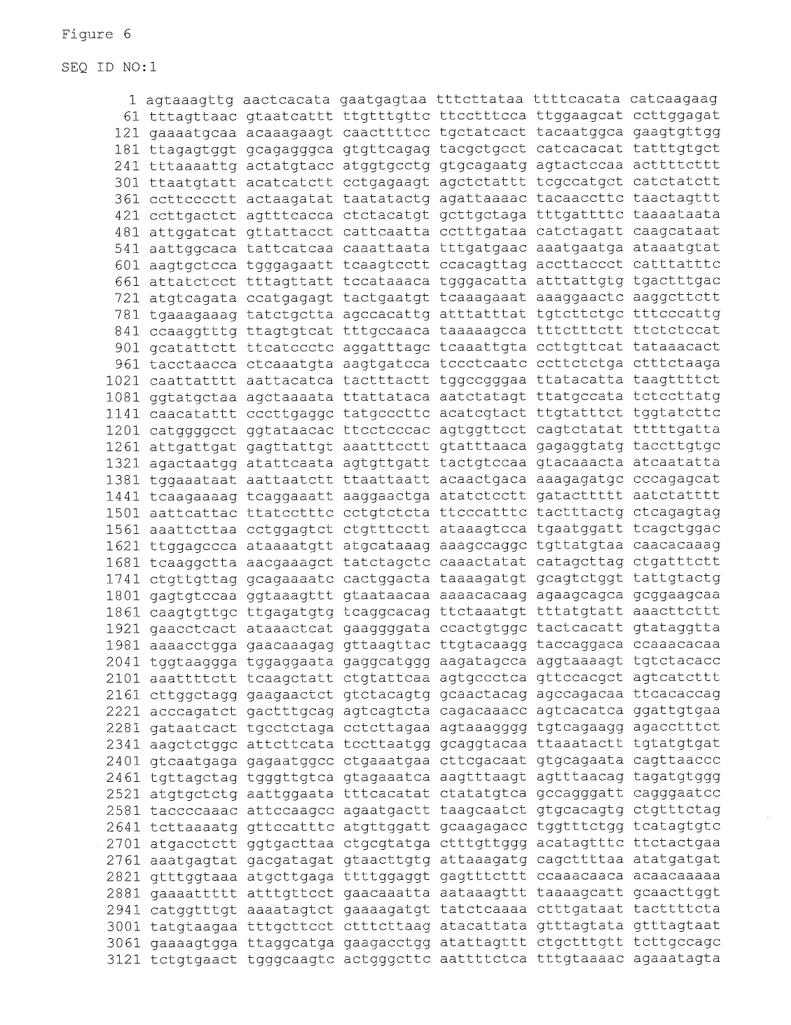

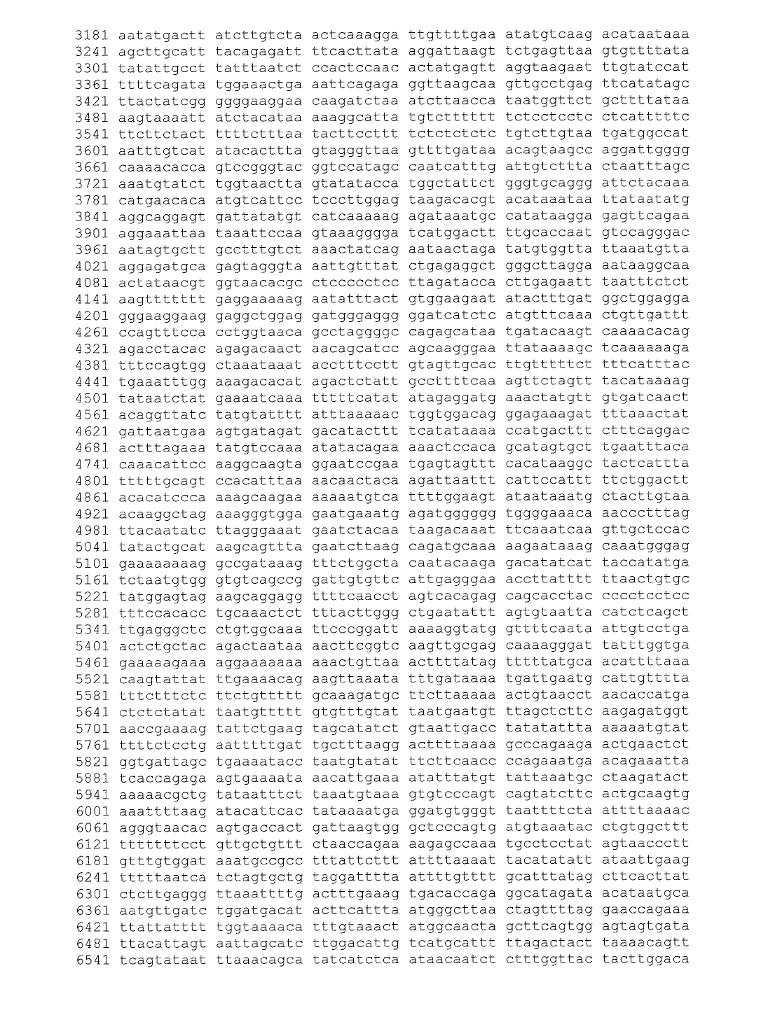

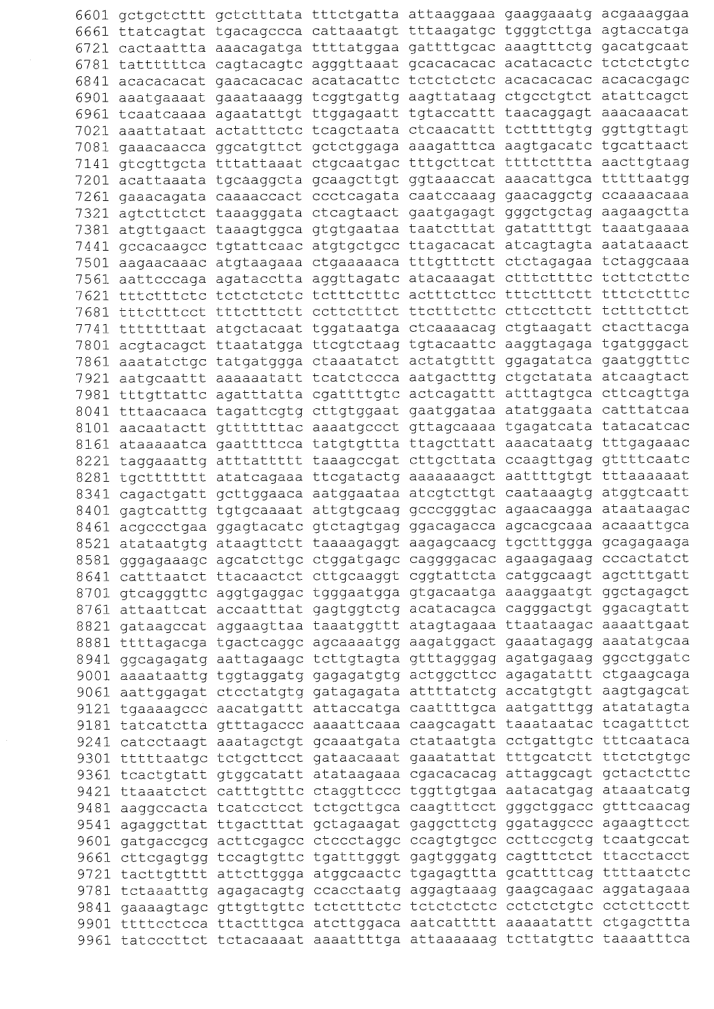

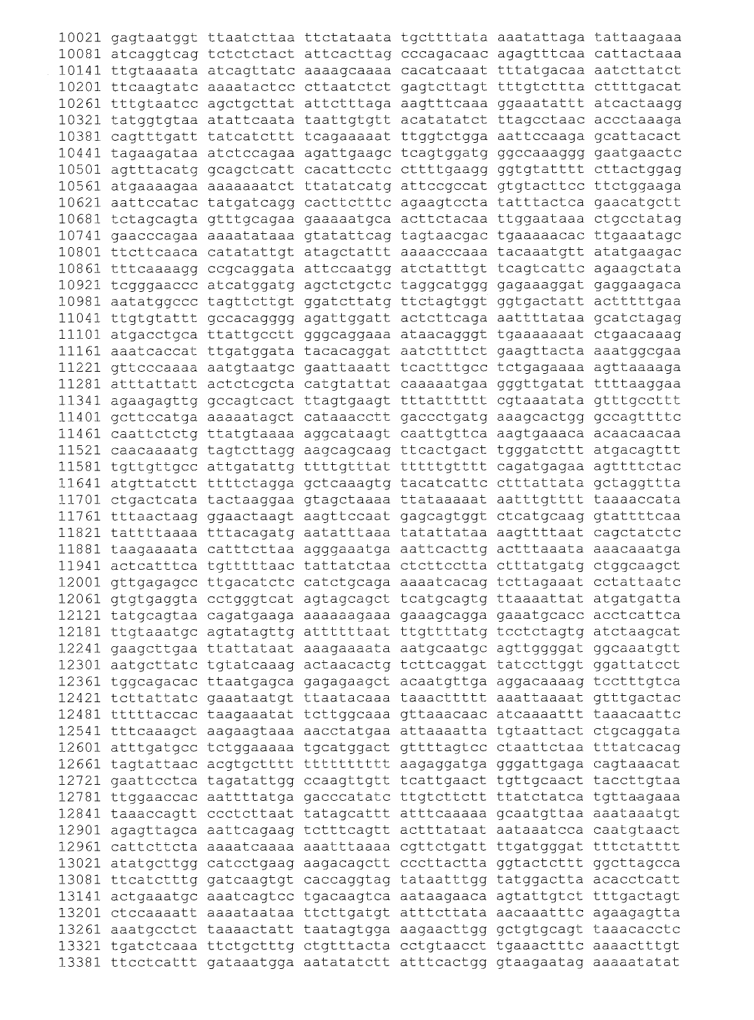

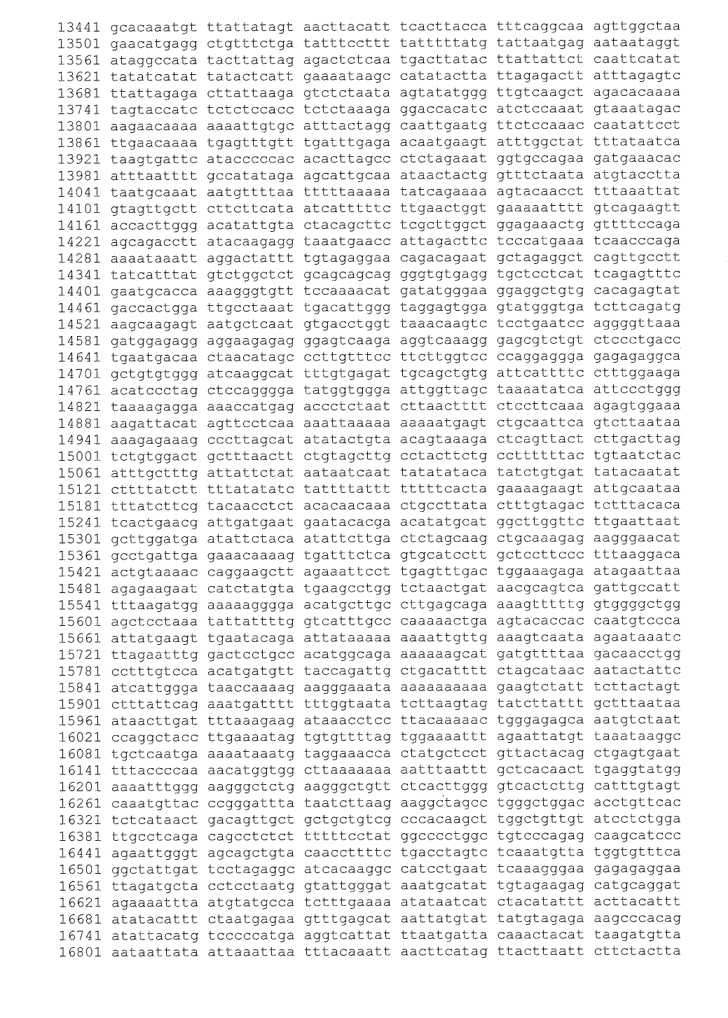

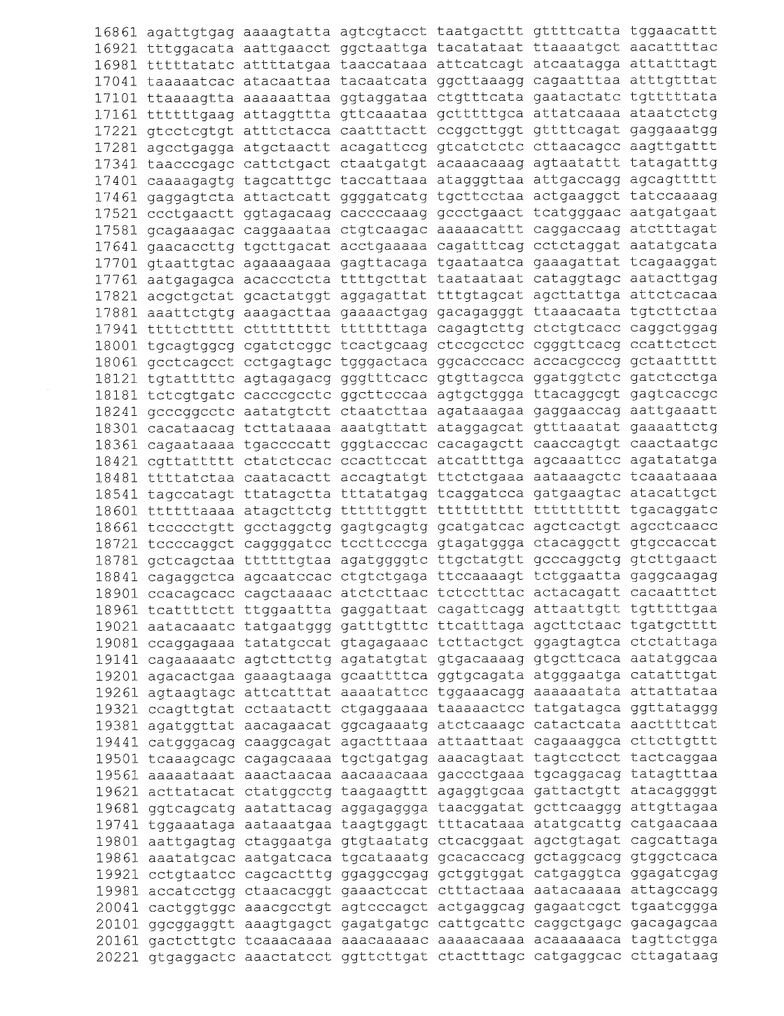

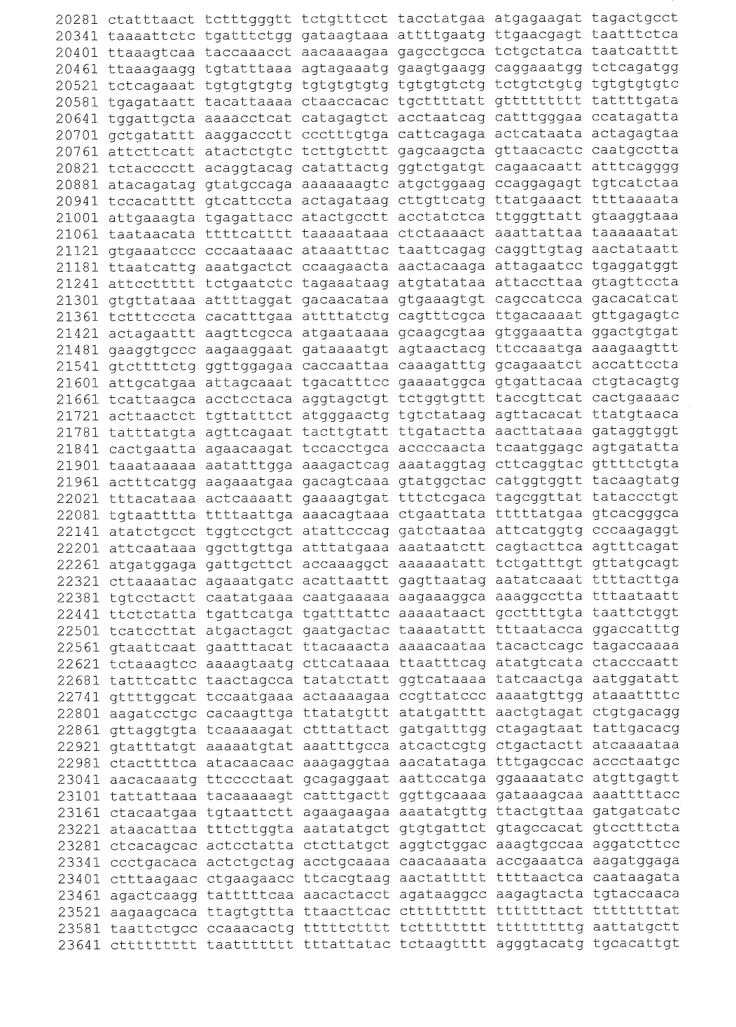

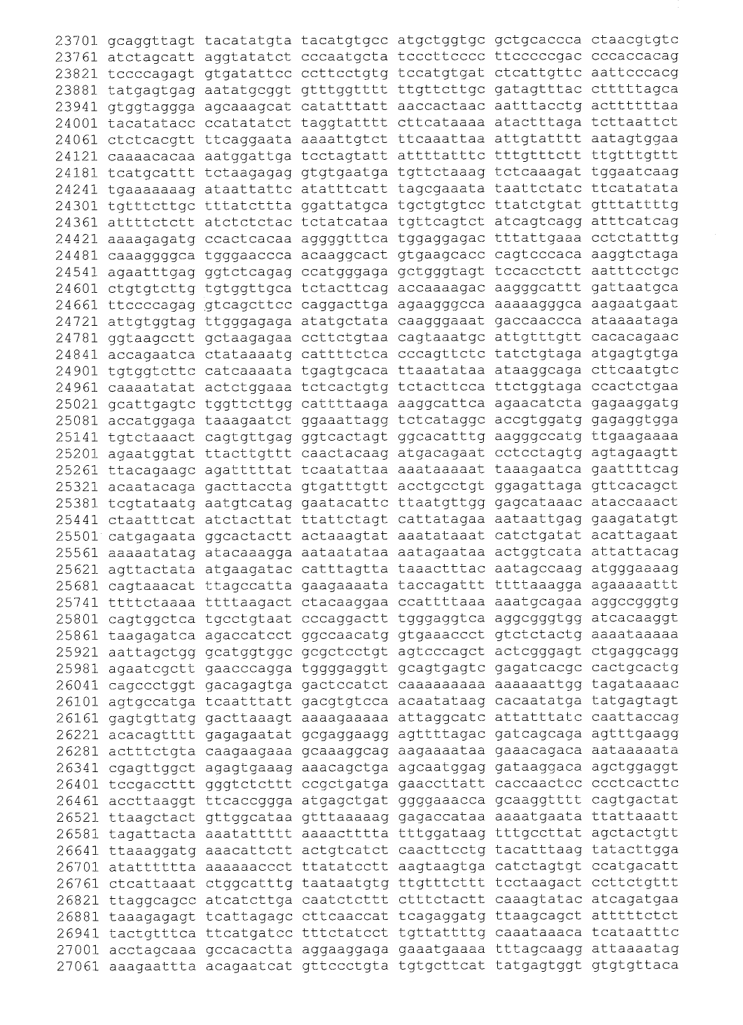

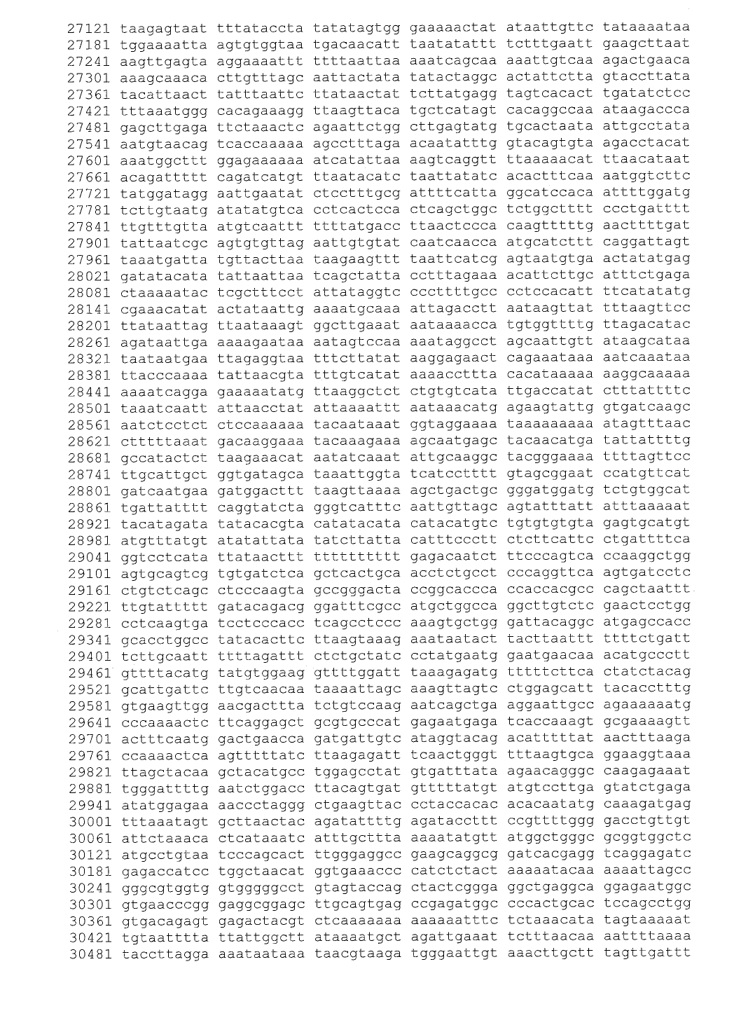

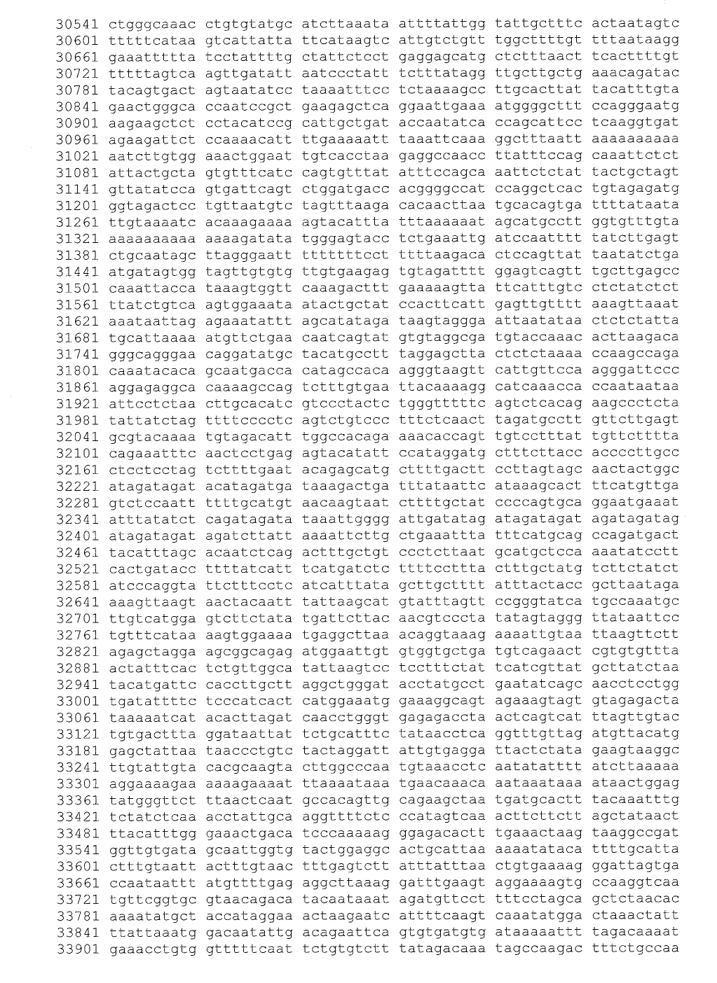

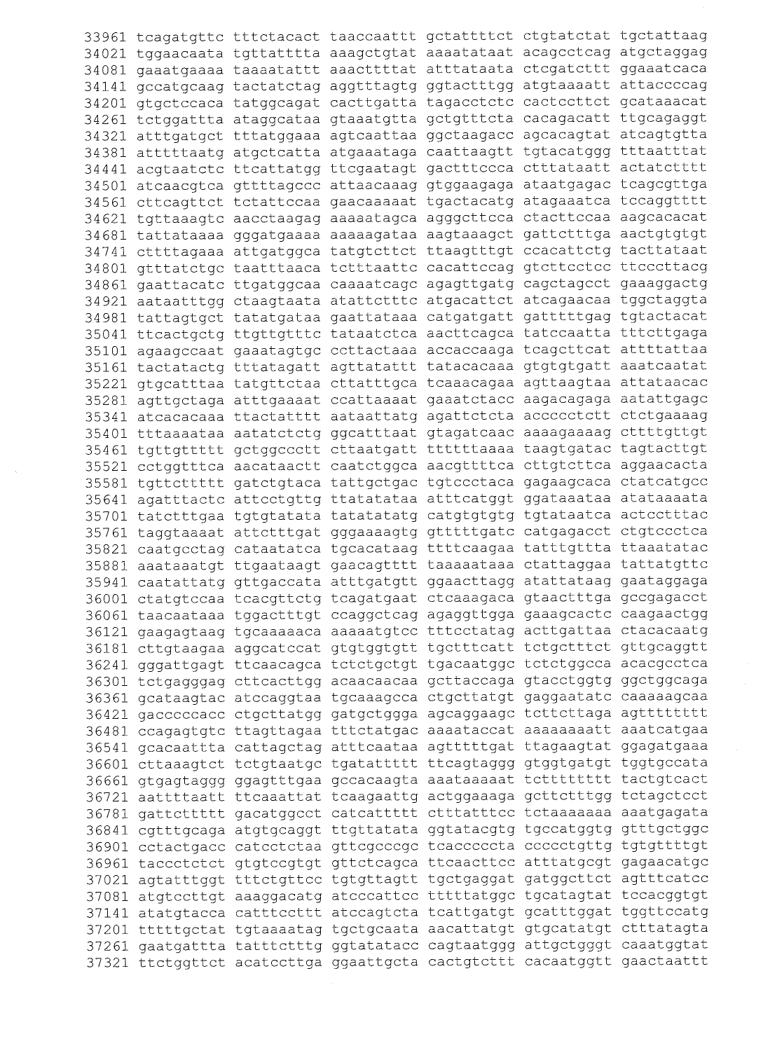

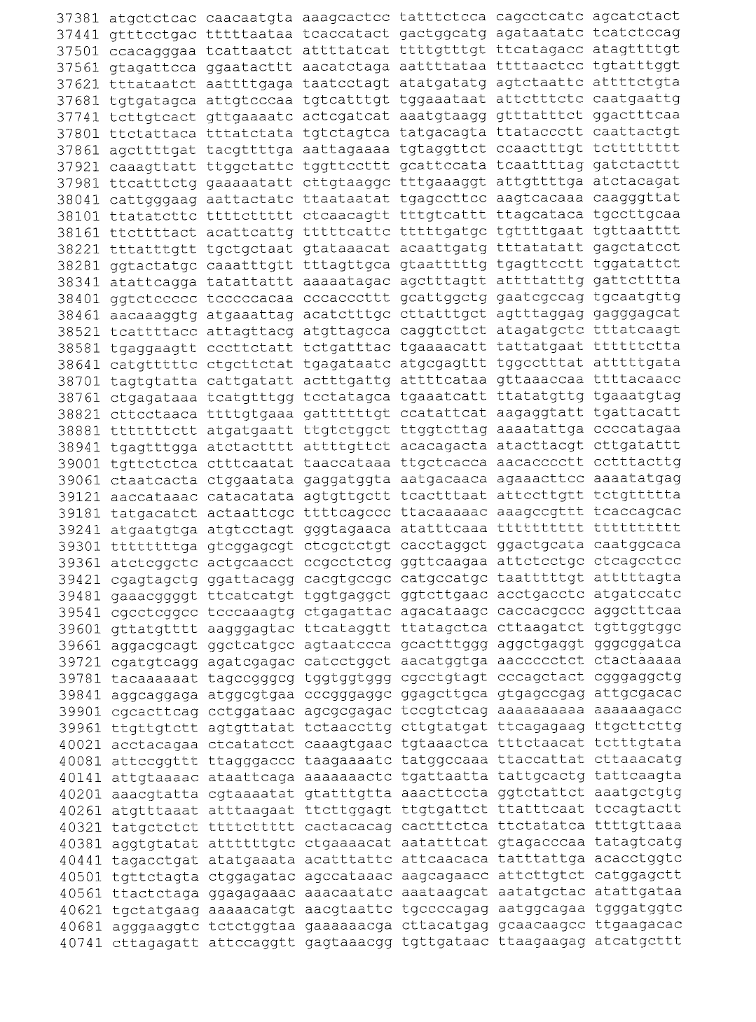

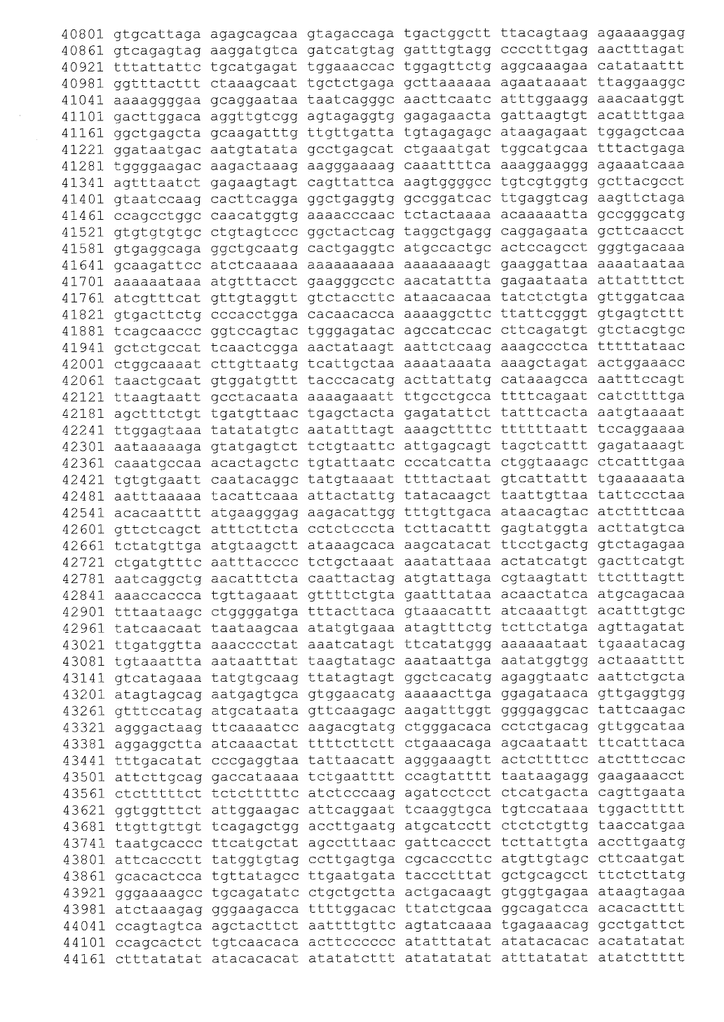

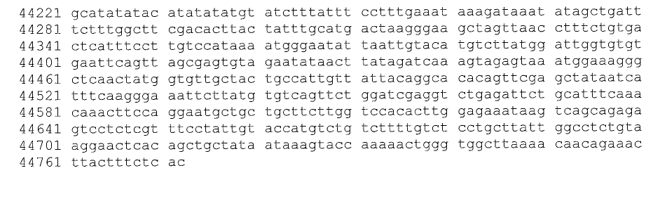

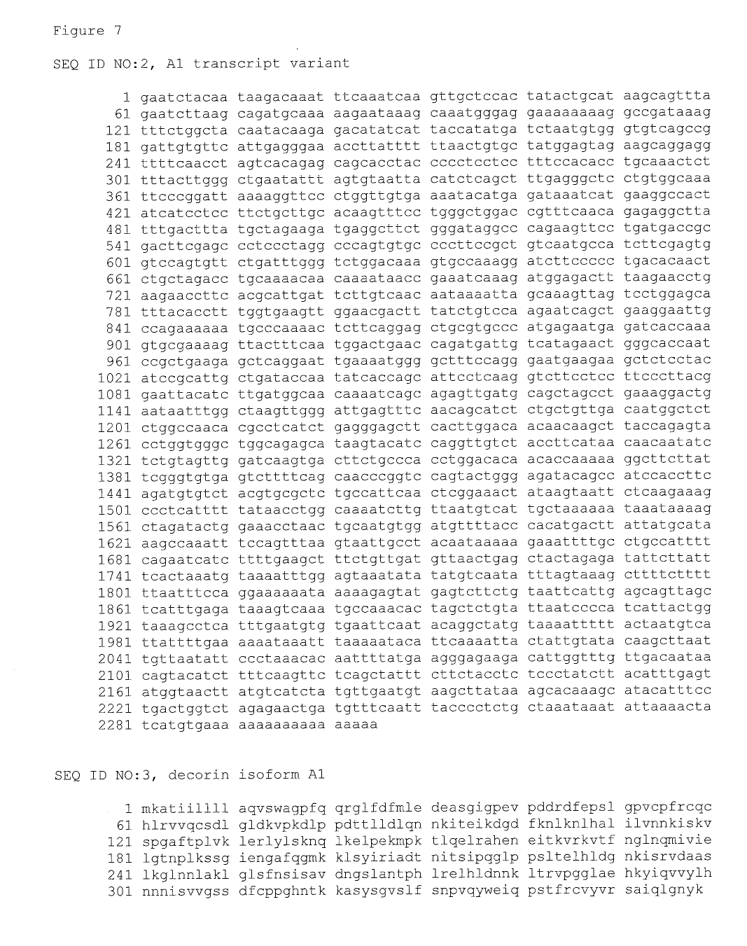

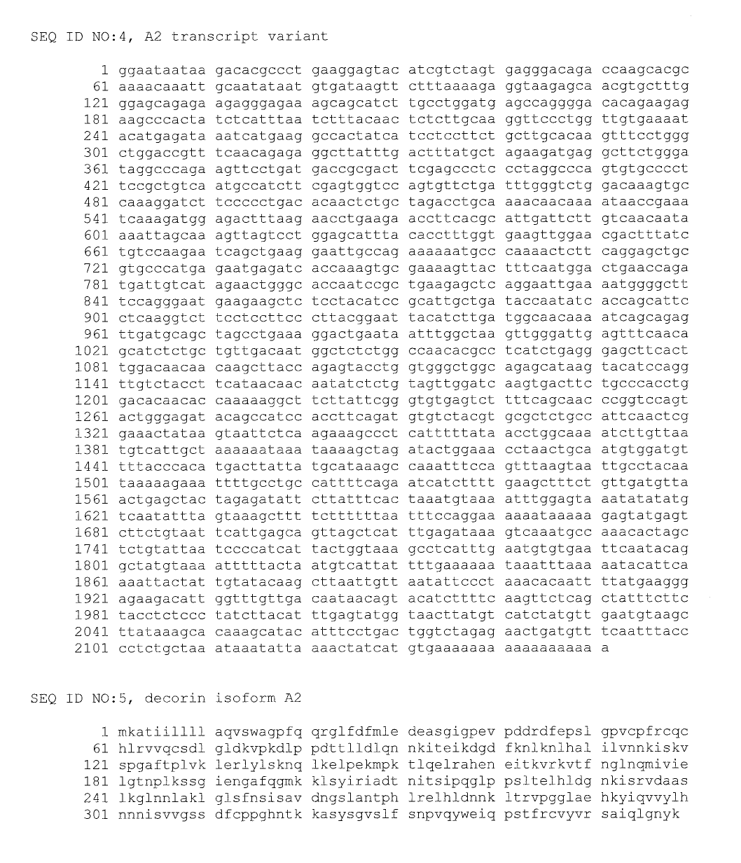

Attenuation of IL-8 production in decorin silenced DOK and SCC25 cells. IL-8 is an important proinflammatory chemokine produced by epithelial cells and is known to be regulated via TLR5 (Yu et al., American journal of physiology. 2003 August; 285(2):G282-90). Therefore, we sought to determine if nuclear decorin silencing-mediated TLR5 down regulation has an effect on IL-8 production in these dysplastic and malignant oral epithelial cells. First, multiplex RT PCR was performed to characterize the effect of decorin silencing on a set of cytokines expression. We did not observe any significant change in IL-10, IL-14, and TGFβ1 between decorin silenced and control or WT DOK or SCC-25 cells (FIG. 4 A). However, IL-8 expression was significantly reduced in nuclear decorin-silenced DOK or SCC-25 cells as compared to the control and WT cells (FIG. 4A). Real-time PCR analysis revealed over 90% reduction in constitutive IL-8 expression in decorin-silenced DOK and about 70% reduction in decorin-silenced SCC-25 cells (FIG. 4B). Constitutive IL-8 production, as measured by ELISA for protein levels, was found to be reduced significantly in decorin-silenced DOK and SCC-25 cells (FIG. 4C). However, as observed with IL-8 expression levels, the effect of decorin silencing on IL-8 production was more pronounced in DOK than in SCC-25 cells. Flagellin is a known ligand for TLR5 and flagellin stimulation of epithelial cells results in increased IL-8 production. To ensure that the IL-8 regulation effects are due to TLR5 down regulation in decorin silenced cells, we determined and compared the levels of IL-8 production upon flagellin stimulation in these cells. Briefly, cells were stimulated with flagellin for 24, 48 and 72 h and 24 h time point was considered optimal for comparing IL-8 production. Consistent with down regulation of TLR5 expression levels as shown previously, we found a significant reduction in flagellin stimulated IL-8 production in decorin silenced cells compared to WT or ctrl-shRNA treated DOK or SCC-35 cells (FIG. 4D). It is interesting to note that SCC-25 cells produce much higher levels of flagellin stimulated IL-8 production than DOK cells.

Decorin silencing mitigates migratory and invasive phenotype of dysplastic and malignant oral epithelial cells. Having determined that nuclear decorin silencing results in reduced TLR5 expression and IL-8 production and based on known pro-invasive functions of IL-8, we next examined whether decorin silencing has any effect on migration and invasion properties of dysplastic and malignant oral epithelial cells. Using an in vitro trans well assay and 10% FBS as a chemo-attractant, we observed a significant suppression of cell migration in both decorin-silenced DOK and SCC-25 cells compared to respective WT or control cells (FIGS. 5A & B). Next, we determined the invasive property of these cells as measured through invasion across a Matrigel™ impregnated porous (8 μm) membrane. Invasive phenotype was observed to be significantly suppressed in decorin-silenced SCC-25 cells and was almost completely abrogated in decorin-silenced DOK cells (FIGS. 5C & D). Similar results were obtained when conditioned media from DOK WT was used as a chemo-attractant (data not shown). However, it is important to note that overall malignant SCC-25 cells have relatively higher migration and invasion rates than the premalignant and dysplastic DOK cells.

### Discussion

Oral cancer is a significant health problem throughout the world. It affects the mucosal lining of the oral tissue including the cheek, floor of mouth, tongue and gums. Decorin is a prototype member of small leucine rich proteoglycans and by binding to and sequestering TGF-β, acts as a natural inhibitor of TGF-β signaling pathways (Yamaguchi et al., Nature. 1990 July. 19; 346(6281):281-4). In our previous studies of oral precancerous and cancerous lesions and cellular models of oral cancer progression, we had demonstrated that decorin is aberrantly expressed and localized in the dysplastic and malignant oral epithelial cells (Banerjee et al., Cancer research. 2003 November. 15; 63(22):7769-76). In the present study, we have identified a role of nuclear localized decorin in innate immune receptor expression, chemokine production, migration, and invasion in oral cancer progression from premalignant stages. We investigated the role of nuclear localized decorin by a functional genomics approach through stably silencing decorin in these cells with a specific shRNAi plasmid vector.

In most of the studies, that have analyzed the role of decorin in tumour physiology, decorin is not expressed in the cancerous epithelial tissue as has been demonstrated in colon, pancreas, prostate, lung, ovarian, and breast cancer (Iozzo and Cohen, Experientia. 1993 May 15; 49(5):447-55, McDoniels-Silvers et al., Clin Cancer Res. 2002 April; 8(4):1127-38, Shridhar et al., Cancer research. 2001 August. 1; 61(15):5895-904, Troup et al., Clin Cancer Res. 2003 January; 9(1):207-14). Rather, it is expressed in the tumor stroma and has been shown to inhibit tumour cell growth and trigger apoptosis (De Luca et al., The Journal of biological chemistry. 1996 August. 2; 271(31):18961-5, Nash et al., Cancer research. 1999 Dec. 15; 59(24):6192-6, Seidler et al., The Journal of biological chemistry. 2006 Sep. 8; 281(36):26408-18). It has been suggested that tumour growth inhibition in the afore-mentioned cancers might be regulated through decorin binding and inhibition of the epidermal growth factor receptor (EGFR). However, we show here in our studies that nuclear localized decorin in oral dysplastic and malignant epithelial cells did not have any effect on cell proliferation. This might be due to sequestration of decorin in the nucleus and inability to interact with membrane epidermal growth factor receptors. Our finding is also consistent with studies in osteosarcoma, where cancerous cells were not sensitive to decorin-induced growth arrest (Zafiropoulos et al., Connective tissue research. 2008; 49(3):244-8).

Besides decorin's function as a competing ligand for EGFR, it has a prominent role in immune regulation as it acts as a physiological inhibitor of TGF-β signaling and activity. TGF-β is an immunosuppressive molecule and plays a central role in maintaining normal immune function. Lack of TGF-β has been associated with aberrant toll-like receptor expression (McCartney-Francis et al., J. Immunol. 2004 March. 15; 172(6):3814-21). In addition, TGF-β has been shown to inhibit TLR2 and TLR4 expression in odontoblasts (Horst et al., Journal of dental research. 2009 April; 88(4):333-8). Our data here indicates that nuclear decorin knock down, leads to suppression of TLR5 expression. Decorin acts as an inhibitor of TGF-β in the extracellular milieu and in its absence unabated signaling may cause premalignant lesions to progress, through multitude of tumour promoting activities known for TGF-β. However, in our study decorin knock down did not have any effect on the expression of TLR1, TLR2, TLR3 and TLR6. Only TLR 5 seemed to be co-regulated at transcriptional level by nuclear localized decorin. We are pursuing further the mechanistic studies of such TLR5 gene regulation in these decorin silenced cells.

The chemokine IL-8 is the quintessential epithelial proinflammatory gene that drives mucosal inflammation and serves to recruit inflammatory cells to the mucosal surfaces (McCormick et al., The Journal of cell biology. 1993 November; 123(4):895-907, McCormick et al., The Journal of cell biology. 1995 December: 131(6 Pt 1):1599-608). In addition, most primary and metastatic tumours, such as breast, uterine, prostate, colon and pancreatic carcinomas, melanoma, and glioblastoma, are known to constitutively express IL-8 (also termed as CXCL8) (Youngs et al., International journal of cancer. 1997 April. 10; 71(2):257-66, Huang et al., The American journal of pathology. 2002 July; 161(1):125-34, Fasciani et al., Molecular human reproduction. 2000 January; 6(1):50-4, Li et al., Clin Cancer Res. 2001 October; 7(10):3298-304). We demonstrate that depletion of nuclear decorin in both premalignant (DOK) and malignant (SCC-25) oral epithelial cells, results in reduced IL-8 production. Therefore implications for targeting decorin in oral cancer progression are very promising. Recently, there has been increasing evidence that chemokines have a role in tumour biology. Chemokines were first described as small peptides controlling cell migration, especially that of leukocytes during inflammation and immune response. Since then, a broad spectrum of biological activities has been described as chemokine-regulated tumorigenesis (Murphy et al., The New England journal of medicine. 2001 Sep. 13; 345(11):833-5, Homey et al., Nature reviews. 2002 March; 2(3):175-84, Strieter, Nature immunology. 2001 April; 2(4):285-6) that effect tumors and their microenvironment. The role of chemokines in tumor biology is important because these peptides may influence tumour growth, invasion, and metastasis. We have shown in this study that levels of IL-8 and consequent invasion index is paramount in oral cancer progression and ablating nuclear decorin related activity in the premalignant and malignant oral cells may be a way of controlling development of oral cancer.

Deciphering biological activity of decorin is complex because of the fact that it regulates multiple processes in the extracellular matrix as well as variable functions in different tumor cells. Together, results from our study suggest the importance of decorin in oral cancer as an important therapeutic target, as it modulates migration and invasion of premalignant and malignant oral epithelial cells. Further mechanistic studies are warranted to know how exactly the gene expression of TLR5 is regulated by nuclear localization of decorin in these cells. Studies in our laboratory are underway in this direction and which will shed some light on additional biological aspects of nuclear localized decorin in oral cancer progression.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for decreasing expression of a decorin polypeptide in a cell comprising:
contacting an oral epithelial cell with an effective amount of a polynucleotide, wherein the polynucleotide comprises a nucleotide sequence substantially identical to, or substantially complementary to, consecutive nucleotides of a target mRNA encoding a decorin polypeptide; and
measuring the decorin polypeptide in the cell, wherein the cell comprising the polynucleotide has less decorin polypeptide when compared to decorin polypeptide present in a corresponding control cell that does not comprise the polynucleotide.

2. The method of claim 1 wherein the oral epithelial cell is a dysplastic cell.

3. The method of claim 1 wherein the oral epithelial cell is a carcinoma cell.

4. The method of claim 1 wherein the oral epithelial cell is a malignant cell.

5. The method of claim 1 wherein the oral epithelial cell is ex vivo.

6. The method of claim 1 wherein the oral epithelial cell is a human cell.

7. The method of claim 1 wherein the polynucleotide is double stranded.

8. The method of claim 7 wherein the double stranded polynucleotide comprises ribonucleotides.

9. The method of claim 7 wherein the double stranded polynucleotide consists of ribonucleotides.

10. The method of claim 7 wherein the double stranded polynucleotide comprises deoxynucleotides.

11. The method of claim 7 wherein the double stranded polynucleotide consists of deoxynucleotides.

12. The method of claim 11 wherein the double stranded polynucleotide is present in a vector.

13. The method of claim 1 wherein the polynucleotide comprises one or more modifications.

14. The method of claim 1 wherein the modifications are selected from a modified nucleic acid sugar, a modified base, a modified backbone, or a combination thereof.

15. The method of claim 8 wherein the double stranded polynucleotide comprises a nucleotide sequence of between 19 and 29 nucleotides.

16. The method of claim 1 wherein the target mRNA is an A1 transcript variant or an A2 transcript variant.

17. The method of claim 16 wherein the polynucleotide comprises a nucleotide sequence substantially identical to, or substantially complementary to, consecutive nucleotides in exon 1, exon 2, exon 3a, exon 4, exon 5, exon 6, exon 7, exon 8, or exon 9.

18. The method of claim 16 wherein the polynucleotide comprises a nucleotide sequence substantially identical to, or substantially complementary to, consecutive nucleotides spanning exons 1 and 2, exons 2 and 3a, exons 3a and 4, exons 4 and 5, or exons 5 and 6.

19. The method of claim 1 wherein the polynucleotide comprises at least 19 consecutive nucleotides selected from GAAGAACCTTCACGCATTGAT (SEQ ID NO:6), or the complement thereof.

20. The method of claim 1 wherein the polynucleotide completely inhibits expression of the decorin polypeptide.

21. The method of claim 8 wherein the double stranded RNA comprises a single strand comprising self-complementary portions.

22. The method of claim 8 wherein the double stranded RNA comprises two separate complementary strands.

23. The method of claim 1 further comprising measuring the motility of the cell.

24. The method of claim 8 wherein motility of the oral epithelial cell is decreased when compared to the control cell.

25. The method of claim 1 wherein the decorin polypeptide is associated with the nucleus of the oral epithelial cell.

26. The method of claim 1 wherein expression of a Toll like receptor 5, interleukin-8, or a combination thereof, by the oral epithelial cell is decreased when compared to the control cell.

27. A double stranded RNA polynucleotide that inhibits expression of a polynucleotide encoding a decorin polypeptide, wherein the double stranded RNA polynucleotide comprises a nucleotide sequence substantially identical to, or complementary to, consecutive nucleotides of exon 1, exon 2, exon 3a, or exon 5.

28. A double stranded RNA polynucleotide that inhibits expression of a polynucleotide encoding a decorin polypeptide, wherein the double stranded RNA polynucleotide comprises a nucleotide sequence substantially identical to, or complementary to, consecutive nucleotides spanning exons 1 and 2, exons 2 and 3a, exons 3a and 4, exons 4 and 5, or exons 5 and 6.

29. The double stranded RNA polynucleotide of claim 2 wherein the nucleotide sequence is substantially identical to at least 19 consecutive nucleotides selected from GAAGAACCTTCACGCATTGAT (SEQ ID NO:6).

30. A method for identifying an agent that alters the distribution of decorin polypeptide in a cell comprising:
contacting an oral epithelial cell with an agent,
incubating the oral epithelial cell and the agent under conditions suitable for growth of the oral epithelial cell; and
measuring the decorin poylpeptide present in the nucleus of the oral epithelial cell, wherein the oral epithelial cell contacted with the agent having less decorin polypeptide present in the nucleus when compared to decorin polypeptide present in the nucleus of a corresponding control cell that does not comprise the agent indicates the agent alters the distribution of decorin polypeptide in a cell.

31. A method for determining a prognosis for oral cancer in a subject comprising:
providing an oral epithelial cell from a subject;
contacting the cell with a compound that binds decorin polypeptide; and
detecting the presence of a decorin polypeptide in an oral epithelial cell, wherein the presence of the polypeptide associated with the nucleus or cytoplasm of the oral epithelial cell indicates a prognosis of increased risk of oral-cancer, and wherein the absence of the polypeptide associated with the nucleus or cytoplasm of the oral epithelial cell indicates a prognosis of decreased risk of oral cancer.

32. The method of claim 31 wherein the compound is an antibody that specifically binds to the polypeptide.

33. The method of claim 31 wherein the polypeptide is encoded by an A1 transcript variant or an A2 transcript variant.
